SEQUENCE LISTING (22 sequences)

SEQ ID NO: 1 - DNA, 870 bp, Aequorea victoria
atggctcgtctttcttttgtttctcttctttctctgtcactgctcttcgggcagcaagcagtcagagctcagaattacaccatggtgagcaagggcgaggagctgttcaccggggtggtgcccatcctggtcgagctggacggcgacgtaaacggccacaagttcagcgtgtccggcgagggcgagggcgatgccacctacggcaaggactgcctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctatacaagtgggcgcgccactcgagacgaatcactagtgaattcgcggccgcctgcaggtcgaggtttgcagcagagtag

SEQ ID NO: 2 - DNA, 57 bp, Homo sapiens
atgaagttatgggatgtcgtggctgtctgcctggtgctgctccacaccgcgtccgcc

SEQ ID NO: 3 - protein, 19 aa, Homo sapiens
MKLWDVVAVCLVLLHTASA

SEQ ID NO: 4 - DNA, 2550 bp, Mus musculus
atgaagcgagcagctgcaaagcatctaatagaacgctactaccatcagttaactgagggctgtggaaatgaggcctgcacgaatgagttttgtgcttcctgtccaacttttcttcgtatggataacaatgcagcagctattaaagcccttgagctttataaaattaatgcaaaactctgtgatcctcatccctccaagaaaggagcaagctcagcttaccttgagaactcaaaaggtgcatctaacaactcagagataaaaatgaacaagaaggaaggaaaagattttaaagatgtgatttacctaactgaagagaaagtatatgaaatttatgaattttgtagagagagtgaggattattcccctttaattcgtgtaattggaagaatattttctagtgctgaggcactggttctgagctttcggaaagtcaaacagcacacaaaggaggaattgaaatctcttcaagaaaaggatgaagacaaggatgaagatgaaaaggaaaaagctgcatgttctgctgctgctatggaagaagactcagaagcatcttcttcaaggatgggtgatagttcacagggagacaacaatgtacaaaaattaggtcctgatgatgtgactgtggatattgatgctattagaagggtctacagcagtttgctcgctaatgaaaaattagaaactgccttcctgaatgcacttgtatatctgtcacctaacgtggaatgtgatttgacatatcataatgtgtatactcgagatcctaattatctcaatttgttcattattgtaatggagaatagtaatctccacagtcctgaatatctggaaatggcgttgccattattttgcaaagctatgtgtaagctaccccttgaagctcaaggaaaactgattaggctgtggtctaaatacagtgctgaccagattcggagaatgatggaaacatttcagcaacttattacctacaaagtcataagcaatgaatttaatagccgaaatctagtgaatgatgatgatgccattgttgctgcttcaaagtgtttgaaaatggtttactatgcaaatgtagtgggaggggatgtggacacaaatcataatgaggaagatgatgaagaacccatacctgagtccagcgaattaacacttcaggagcttctgggagatgaaagaagaaataagaaaggtcctcgagtggatccactagaaaccgaacttggcgttaaaactctagactgtcgaaaaccacttatctcctttgaagaattcattaatgaaccactgaatgatgttctagaaatggacaaagattatacctttttcaaagttgaaacagagaacaaattctcttttatgacatgtccctttatattgaatgctgtcacaaagaatctgggattatattatgacaatagaattcgcatgtacagtgaaagaagaatcactgttctttacagcctagttcaaggacagcagttgaatccgtatttgagactcaaagtcagacgtgaccatattatagatgatgcactggtccggctagagatgattgctatggaaaatcctgcagacttgaagaagcagttgtatgtggaatttgaaggagaacaaggagtaatgagggaggcgtttccaaagagttttttcagttgggttgtggaggaaatttttaatccaaatattggtatgttcacatatgatgaagctacgaaattattttggtttaatccatcttcttttgaaactgagggtcaggtttactctgattggcatatcctgggtctggctatttacaataattgtatactggatgtccattttcccatggttgtatacaggaagctaatggggaaaaaaggaacctttcgtgacttgggagactctcacccagttttatatcagagtttaaaggatttattggaatatgaagggagtgtggaagatgatatgatgatcactttccagatatcacagacagatctttttggtaacccaatgatgtatgatctaaaagaaaatggtgataaaattccaattacaaatgaaaacaggaaggaatttgtcaatctctattcagactacattctcaataaatctgtagaaaaacaattcaaggcatttcgcagaggttttcatatggtgactaatgaatcgcccttaaaatacttattcagaccagaagaaattgaattgcttatatgtggaagccggaatctagatttccaggcactagaagaaactacagagtatgacggtggctatacgagggaatctgttgtgattagggagttctgggaaattgttcattcgtttacagatgaacagaaaagactctttctgcagtttacaacaggcacagacagagcacctgttggaggactaggaaaattgaagatgattatagccaaaaatggcccagacacagaaaggttacctacatctcatacttgctttaatgtccttttacttccggaatattcaagcaaagaaaaacttaaagagagattgttgaaggccatcacatatgccaaaggatttggcatgctgtaa

SEQ ID NO: 5 - DNA, 78 bp, Mus musculus
atggccctgttggtgcacttcctacccctgctggccctgcttgccctctgggagcccaaacccacccaggcttttgtc

SEQ ID NO: 6 - protein, 26 aa, Mus musculus
MALLVHFLPLLALLALWEPKPTQAFV

SEQ ID NO: 7 - DNA, 33 bp, Human Immunodeficiency Virus
tacggcagaaagaagaggaggcagagaaggaga

SEQ ID NO: 8 - protein, 11 aa, Human Immunodeficiency Virus
YGRKKRRQRRR

SEQ ID NO: 9 - DNA, 10893 bp, Homo sapiens
  misc_feature (122)..(122): n is a, c, g or t
  misc_feature (340)..(340): n is a, c, g or t
  misc_feature (1145)..(1145): n is a, c, g or t
  misc_feature (1180)..(1180): n is a, c, g or t
  misc_feature (1257)..(1257): n is a, c, g or t
  misc_feature (5410)..(5410): n is a, c, g or t
  misc_feature (7624)..(7624): n is a, c, g or t
  misc_feature (7644)..(7644): n is a, c, g or t
  misc_feature (7647)..(7647): n is a, c, g or t
  misc_feature (7741)..(7741): n is a, c, g or t
  misc_feature (7836)..(7836): n is a, c, g or t
ggagtagtttactgagccactaatctaaagtttaatactgtgagtgaataccagtgagtacctttgttaatgtggataaccaatacttggctataggaagttttttagttgtgtgttttatnacacgtatttgactttgtgaataattatggcttataatggcttgtctgttggtatctatgtatagcgtttacagtttcctttaaaaaacatgcattgagttttttaatagtccaacccttaaaataaatgtgttgtatggccacctgatctgaccactttctttcatgttgacatctttaattttaaaactgttttatttagtgcttaaatcttgttnacaaaattgtcttcctaagtaatatgtctacctttttttttggaatatggaatattttgctaactgtttctcaattgcattttacagatcaggagaacctcagtctgacgacattgaagctagccgaatgtaagtgtaacttggttgagactgtggttcttattttgagttgccctagactgctttaaattacgtcacattatttggaaataatttctggttaaaagaaaggaatcatttagcagtaaatgggagataggaacatacctactttttttcctatcagataactctaaacctcggtaacagtttactaggtttctactactagatagataaatgcacacgcctaaattcttagtctttttgcttccctggtagcagttgtagggaaatagggaggttgaggaaagagtttaacagtctcaacgcctaccatatttaaggcatcaagtactatgttatagatacagagatgcgtaataattagttttcaccctacagaaatttatattatactcaagagtgaaagatgcagaagcaaataatttcagtcactgaggtagaatggtatccaaaatacaatagtaacatgaaggagtactggagtaccaggtatgcaataggaatctagtgtagatggcagggaagtaagagtggccaggaaatgctaagttcagtcttgaaatgtgactgggaatcaggcagctatcaactataagtcaaatgtttacaagctgttaaaaatgaaatactgattatgtaaaagaaaaccggattgatgctttaaatagactcattttcntaatgctaatttttaaaatgatagaatcctacaantcttagctgtaaaccttgtgatttttcagctgttgtactaaacaacttaagcacatataccatcagacaagcccccntccccccttttaaaccaaaggaatgtatactctgttaatacagtcagtaagcattgacattctttatcataatatcctagaaaatatttattaactatttcactagtcaggagttgtggtaaatagtgcatctccattttctacttctcatcttcatacacaggttaatcacttcagtgcttgactaacttttgccttgatgatatgttgagctttgtacttgagagctgtactaatcactgtgcttattgtttgaatgtttggtacaggaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaaggtaagatgttttattttcaattgagaattgttgcctgaaaaccatgtgggagatttaaatgtattagtttttatttgttttttcttctgtgacataaagacattttgatatcgtagaaccaattttttattgtggtaacggacaggaataataactacattttacaggtctaatcattgctaattagaagcagatcatatgccaaaagttcatttgttaatagattgatttgaactttttaaaattcttaggaaaaatgtattaagtggtagtgaatctccaaaactatttaagagctgtattatgattaatcagtacatgacatattggttcatatttataattaaagctatacattaatagatatcttgattataaagaaagtttaaactcatgatcttattaagagttatacattgttgaaagaatgtaaaagcatgggtgaggtcattggtataggtaggtagttcattgaaaaaaataggtaagcattaaattttgtttgctgaatctaagtattagatactttaagagttgtatatcataaatgatattgagcctagaatgtttggctgttttacttttagaactttttgcaacagagtaaacatacatattatgaaaataaatgttctcttttttcctctgattttctagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccgggtaagttgggctgctagattaaaaacctaataatggggatatcatgatacagttcagtgaattcattttaaaagtgactgaaaaaaatgataccatatagcataggaacacatggacatttctgatcttatataagtattatacttttgttgttcctgtgcaagtttatagatgtgttctacaaagtatcggttgtattatataatggtcatgctatctttgaaaaagaatgggttttctaaatcttgaaaactaaatccaaagtttctttcattcagaagagaatagagtgttggacaaagaccagaacaagagaaatgtggagatacccaataataagtgtggatgtgcagtcttgaactgggagtaatggtacagtaaaaccataccataaaattataggtagtgtccaaaaaattccatcgtgtaaaattcagagttgcattattgtggacttgaagaagcagttgtatgtgggacggtatcgataagcttgatatcgaattcctgcagcccgggggatccactagtgtggtaattaatactaagtcttactgtgagagaccataaactgctttagtattcagtgtatttttcttaattgaaatatttaacttatgacttagtagatactaagacttaacccttgagtttctattctaataaaggactactaatgaacaattttgaggttagacctctactccattgtttttgctgaaatgatttagctgcttttccatgtcctgtgtagtccagacttaacacacaagtaataaaatcttaattaattgtatgttaatttcataacaaatcagtaaagttagctttttactatgctagtgtctgttttgtgtctgtctttttgattatctttaagactgaatctttgtcttcactggctttttatcagtttgctttctgtttccatttacatacaaaaagtcaaaaatttgtatttgtttcctaatcctactccttgtttttattttgtttttttcctgatactagcaatcatcttcttttcatgtttatcttttcaatcactagctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtaaatacattagtaatgtgattatggtgtcgtatcatcttttgagttagttatttgtttatcttactttgtaaatattttcagctatgaagagcagcaaaagaaggatttggtatggattacccagaatcacacatcatgactgaatttgtaggttttaggaactgatttgtatcactaatttattcaaattcttttatttcttagaaggaatattctaatgaaggaaattatctctttggtaaactgaattgaaagcactttagaatggtatattggaacagttggagggatttctttgctttttgttgtctaaaaccatcatcaaactcacggttttcctgacctgtgaacttcaaagaacaatggtttgaagagtattgagagactgtctcacaagtatgtcatgctcaaagttcagaaacactagctgatatcacattaattaggtttatttgctataagatttcttggggcttaatatangtagtgttcccccaaactttttgaactccagaactcttttctgccctaacagtagctactcaggagctgaggcaggagaattgtttgaacctaggaggcagaggttgcagtgagctgagatcgtgccactccagcccacccctgggtaacagagcgagactccatctcaaagaaaaaaatgaaaaattgttttcaaaaatagtacgtgtggtacagatataagtaattatatttttataaatgaaacactttggaaatgtagccattttttgtttttttatgtttatttttcagctatgggtggataaagcatgaatataacttttcttatgtgttagtagaaaattagaaagcttgaatttaattaacgtatttttctacccgatgccaccaaattacttactactttattcctttggcttcataaaattacatatcaccattcaccccaatttatagcagatatatgtggacattgttttctcaagtgctaatataatagaaatcaatgttgcatgcctaattacatatattttaaatgttttatatgcataattattttaagtttatatttgtattattcatcagtccttaataaaatacaaaagtaatgtatttttaaaaatcatttcttataggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagtaagttctttgtcatttttttaattcagtctcttagattttatttaaatgcaaaaatttaatttatgtcaaaattttaaagtttttgtttagaatctttgttgatactcttatcaataagataaaaatgttttaatctgaccgaagtaccagaaacacttaaaaactcaaagggggacatttttatatattgctgtcagcacgaagctttcgtaagattgatttcatagagaagtgtttctaaacattttgtttgtgttttagtgaaatcttaagagataggtaaaaatcagagtagccctggctaagggtcttggtagttacaacgagtgtgcctgctcctaccacccccacccccaccttgagacaccacagaatttctcatagagcacagtgtgaattctattgctaaattggtggtatggggtttctcagcagagaatgggacatcacagtgactgacaatctttcttttataggttggaaactatttgggggactggagggatactgtctacactttttacaatttttattgataagatttttgttgtcttctaagaagagtgatataaattatttgttgtattttgtagttctatggtggcctcaatttaccatttctggttgctaggttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggtaataaatgtttttatgtcacattttgtctcttcattaacactttcaaagcatgtatgcttataatttttaaagaagtatctaatatagtctgtacaaaaaaaaaacaagtaactaagtttatgtaaatgctagagtccacttttctaaatcttggatataagttggtatgaaagcacacagttgggcactaaagccccttttagagaaagaggacatgaagcaggagatagttaatagctaagtgtggttgtagtataaagcaagaagcagggtgtttcttgtattaagctgtaagcaggaacctcatgattaaggtctttatcacagaacaaataaaaattacatttaatttacacatgtatatcctgtttgtgataaaaatacatttctgaaaagtatactttacgtcagatttgggttctattgactaaaatgtgttcatcgggaatgggaataacccagaacataacaagcaaaaaattatgacaaatatatagtatacctttaagaaacatgtttatattgatataattttttgattaaatattatacacactaagggtacaangcacattttccttttatganttngatacagtagtttatgtgtcagtcagatacttccacatttttgctgaactggatacagtaagcagcttaccaaatattctatggtagaaaactnggacttcctggtttgcttaaatcaaatatattgtactctcttaaaacggttggcatttataaatagatggatacatggtttaaatgtgtctgttnacatacctagttgagagaacctaaagaattttctgcgtctccagcatttatattcagttctgtttaatacattatcgaaattgacatttataagtatgacagttttgtgtatatggccttttcatagcttaatattggctgtaacagagaattgtgaaattgtaagaagtagttttctttgtaggtgtaaaattgaatttttaagaatattcttgacagttttatgtatatggccttttcatagcttaatattggctataacagagaattgtgaaattgttaagaagtaggtgtaaaattgaatttttaagaatattcttgaatgtttttttcttggaaaaattaaaaagctatgcagcccaataacttgtgttttgtttgcatagcatattataagaagttcttgtgattaatgttttctacaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccgggtaagaaagcaggtgtctgcaaaaagtcatgtatcgatttattgtttgtaatgatacagtagtatagcagataactaagacatattttcttgaatttgcagaatctagatttccaagcactagaagaaactacagaatatgacggtggctataccagggactctgttctgattaggtgaggtacttagttcttcagaggaagatttgattcaccaaaggggtgtgtgattttgcttcagacctttatctctaggtactaattcccaaataagcaaactcacaaattgtcatctatatacttagatttgtatttgtaatataatcaccatttttcagagctaatcttgtgatttatttcatgaatgaagtgttgttatatataagtctcatgtaatctcctgcatttggcgtatggattatctagtattcctcactggttagagtatgcttactgctggttagaagataattaaaataaggctaccatgtctgcaatttttcctttcttttgaactctgcatttgtgaactgttacatggcttcccaggatcaagcactttttgagtgaaatggtagtcttttatttaattcttaagataatatgtccagatacatactagtatttccattttacaccctaaaaaactaagccctgaattctcacagaaagatgtagaggttcccagttctatctgcttttaaacaaatgcccttactactctactgtctacttctgtgtactacatcatcgtatgtagttgtttgcatttgggccagttggttggggcaggggtctttttttcttttgtcccttaatctgtatcactttttcctcccaaagttgagttaaaggatgagtagaccaggagaataaaggagaaaggataaataaaatatatacccaaaggcacctggagttaatttttccaaatattcatttcagtctttttcaattcataggattttgtcttttgctcattactgactgcataatgtgattataccatagtttaaatagtcacttcctgttactacacacttgggttttctcaattttttactattgtagtactaatattttactatattgtaatctaatccaaatttttacgtattcagagctgttcaggataaatttgcttggaaatttttaaatcaccagaagtgatactatcctgataattaacttccaagttgtctcttaatatagttttaatgcaaatcataagcttatgttagtaccagtcataatgaatgccaaactgaaaccagtattgtattttttctcattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggtaggtaattattaacttgtgactgtatacctaccgaaaaccttgcattcctcgtcacatacatatgaactgtctttatagtttctgagcacattcgtgattttatatacaaatccccaaatcatattagacaattgagaaaatactttgctgtcattgtgtgaggaaacttttaagaaattgccctagttaaaaattattatggggctcacattggtttggaatcaaattagtgtgattcatttacttttttgattcccagcttgttaattgaaagccatataacatgatcatctatttagaatggttacattgaggctcggaagattatcatttgattgtgctagaatcctgttatcaaatcattttcttagtcatattgccagcagtgtttctaataagcatttaagagcacacactttgcagtcttgtaaaacaggtttgagtattttctccaccttagaggaagttacttgacttctcagtgacctaacctctaaagtgcatttactgatgtcctctctgtggttttgttgtggaaagatttagttaaatgaactgtaagaattcagtacctaaaatggtatctgttatgtagtaaaaactcaatggatacagtatcttatcatcgtcactagctttgagtaatttataggataaaggcaacttggtagttacacaacaaaaagtttatgatttgcattaatgtatagtttgcattgcagaccgtctcaactatatacaatctaaaaataggagcatttaattctaagtgtatttcccatgacttacagttttcctgtttttttccccttttctctatttaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttt

SEQ ID NO: 10 - protein, 852 aa, Homo sapiens
MKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 11 - DNA, 72 bp, Homo sapiens
atggccctgtggatgcgcctcctgcccctgctggcgctgctggccctctggggacctgacccagccgcagcc

SEQ ID NO: 12 - DNA, 60 bp, Homo sapiens
atggagacagacacactcctgctatgggtactgctgctctgggttccaggttccactggt

SEQ ID NO: 13 - protein, 11 aa, Human Immunodeficiency Virus
YARKAARQARA

SEQ ID NO: 14 - DNA, 2767 bp, Homo sapiens
acagtatgacatctgatgctggagggtcgcactttcacaaatgagtcagctggtacatggggttatcatcaatttttagctcttctgtctgggagatacaagtttggaagcaatcttggggtacttacccacaaggctggtggagaccagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaggatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaat

SEQ ID NO: 15 - DNA, 5276 bp, Homo sapiens
agccagtcctcccgtcttgcgccgcggccgcgagatccgtgtgtctcccaagatggtggcgctgggctcggggtgactacaggagacgacggggccttttcccttcgccaggacccgacacaccaggcttcgctcgctcgcgcacccctccgccgcgtagccatccgccagcgcgggcgcccgccatccgccgcctacttacgcttcacctctgccgacccggcgcgctcggctgcgggcggcggcgcctccttcggctcctcctcggaatagctcgcggcctgtagcccctggcaggagggcccctcagccccccggtgtggacaggcagcggcggctggcgacgaacgccgggatttcggcggccccggcgctccctttcccggcctcgttttccggataaggaagcgcgggtcccgcatgagccccggcggtggcggcagcgaaagagaacgaggcggtggcgggcggaggcggcgggcgagggcgactacgaccagtgaggcggccgccgcagcccaggcgcgggggcgacgacaggttaaaaatctgtaagagcctgattttagaattcaccagctcctcagaagtttggcgaaatatgagttattaagcctacgctcagatcaaggtagcagctagactggtgtgacaacctgtttttaatcagtgactcaaagctgtgatcaccctgatgtcaccgaatggccacagcttgtaaaagagagttacagtggaggtaaaaggagtggcttgcaggatggagaagctgcaccagtgttattggaaatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaagcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaaggaaaaaaaaagaaaaaatttaaaaaattttaaaaatataacgagggataaatttttggtggtgatagtgtcccagtacaaaaaggctgtaagatagtcaaccacagtagtcacctatgtctgtgcctcccttctttattggggacatgtgggctggaacagcagatttcagctacatatatgaacaaatcctttattattattataattatttttttgcgtgaaagtgttacatattctttcacttgtatgtacagagaggtttttctgaatatttattttaagggttaaatcacttttgcttgtgtttattactgcttgaggttgagccttttgagtatttaaaaaatatataccaacagaactactctcccaaggaaaatattgccaccatttgtagaccacgtaaccttcaagtatgtgctacttttttgtccctgtatctaactcaaatcaggaactgtattttttttaatgatttgcttttgaaacttgaagtcttgaaaacagtgtgatgcaattactgctgttctagcccccaaagagttttctgtgcaaaatcttgagaatcaatcaataaagaaagatggaaggaagggagaaattggaatgttttaactgcagccctcagaactttagtaacagcacaacaaattaaaaacaaaaacaactcatgccacagtatgtcgtcttcatgtgtcttgcaatgaactgtttcagtagccaatcctctttcttagtatatgaaaggacagggatttttgttcttgttgttctcgttgttgttttaagtttactggggaaagtgcatttggccaaatgaaatggtagtcaagcctattgcaacaaagttaggaagtttgttgtttgtttattataaacaaaaagcatgtgaaagtgcacttaagatagagtttttattaattacttacttattacctagattttaaatagacaatccaaagtctccccttcgtgttgccatcatcttgttgaatcagccattttatcgaggcacgtgatcagtgttgcaacataatgaaaaagatggctactgtgccttgtgttacttaatcatacagtaagctgacctggaaatgaatgaaactattactcctaagaattacattgtatagccccacagattaaatttaattaattaattcaaaacatgttaaacgttactttcatgtactatggaaaagtacaagtaggtttacattactgatttccagaagtaagtagtttcccctttcctagtcttctgtgtatgtgatgttgttaatttcttttattgcattataaaataaaaggattatgtatttttaactaaggtgagacattgatatatccttttgctacaagctatagctaatgtgctgagcttgtgccttggtgattgattgattgattgactgattgttttaactgattactgtagatcaacctgatgatttgtttgtttgaaattggcaggaaaaatgcagctttcaaatcattggggggagaaaaaggatgtctttcaggattattttaattaatttttttcataattgagacagaactgtttgttatgtaccataatgctaaataaaactgtggcacttttcaccataatttaatttagtggaaaaagaagacaatgctttccatattgtgataaggtaacatggggtttttctgggccagcctttagaacactgttagggtacatacgctaccttgatgaaagggaccttcgtgcaactgtagtcatcttaaaggcttctcatccactgtgcttcttaatgtgtaattaaagtgaggagaaattaaatactctgagggcgttttatataataaattcgtgaaga

SEQ ID NO: 16 - protein, 875 aa, Homo sapiens
MEKLHQCYWKSGEPQSDDIEASRMKRAAAKHLIERYYHQLTEGCGNEACTNEFCASCPTFLRMDNNAAAIKALELYKINAKLCDPHPSKKGASSAYLENSKGAPNNSCSEIKMNKKGARIDFKDVTYLTEEKVYEILELCREREDYSPLIRVIGRVFSSAEALVQSFRKVKQHTKEELKSLQAKDEDKDEDEKEKAACSAAAMEEDSEASSSRIGDSSQGDNNLQKLGPDDVSVDIDAIRRVYTRLLSNEKIETAFLNALVYLSPNVECDLTYHNVYSRDPNYLNLFIIVMENRNLHSPEYLEMALPLFCKAMSKLPLAAQGKLIRLWSKYNADQIRRMMETFQQLITYKVISNEFNSRNLVNDDDAIVAASKCLKMVYYANVVGGEVDTNHNEEDDEEPIPESSELTLQELLGEERRNKKGPRVDPLETELGVKTLDCRKPLIPFEEFINEPLNEVLEMDKDYTFFKVETENKFSFMTCPFILNAVTKNLGLYYDNRIRMYSERRITVLYSLVQGQQLNPYLRLKVRRDHIIDDALVRLEMIAMENPADLKKQLYVEFEGEQGVDEGGVSKEFFQLVVEEIFNPDIGMFTYDESTKLFWFNPSSFETEGQFTLIGIVLGLAIYNNCILDVHFPMVVYRKLMGKKGTFRDLGDSHPVLYQSLKDLLEYEGNVEDDMMITFQISQTDLFGNPMMYDLKENGDKIPITNENRKEFVNLYSDYILNKSVEKQFKAFRRGFHMVTNESPLKYLFRPEEIELLICGSRNLDFQALEETTEYDGGYTRDSVLIREFWEIVHSFTDEQKRLFLQFTTGTDRAPVGGLGKLKMIIAKNGPDTERLPTSHTCFNVLLLPEYSSKEKLKERLLKAITYAKGFGML

SEQ ID NO: 17 - DNA, 2970 bp, Homo sapiens
tttttccggataaggaagcgcgggtcccgcatgagccccggcggtggcggcagcgaaagagaacgaggcggtggcgggcggaggcggcgggcgagggcgactacgaccagtgaggcggccgccgcagcccaggcgcgggggcgacgacaggttaaaaatctgtaagagcctgattttagaattcaccagctcctcagaagtttggcgaaatatgagttattaagcctacgctcagatcaaggtagcagctagactggtgtgacaacctgtttttaatcagtgactcaaagctgtgatcaccctgatgtcaccgaatggccacagcttgtaaaagatcaggagaacctcagtctgacgacattgaagctagccgaatgaagcgagcagctgcaaagcatctaatagaacgctactaccaccagttaactgagggctgtggaaatgaagcctgcacgaatgagttttgtgcttcctgtccaacttttcttcgtatggataataatgcagcagctattaaagccctcgagctttataagattaatgcaaaactctgtgatcctcatccctccaagaaaggagcaagctcagcttaccttgagaactcgaaaggtgcccccaacaactcctgctctgagataaaaatgaacaagaaaggcgctagaattgattttaaagatgtgacttacttaacagaagagaaggtatatgaaattcttgaattatgtagagaaagagaggattattcccctttaatccgtgttattggaagagttttttctagtgctgaggcattggtacagagcttccggaaagttaaacaacacaccaaggaagaactgaaatctcttcaagcaaaagatgaagacaaagatgaagatgaaaaggaaaaagctgcatgttctgctgctgctatggaagaagactcagaggcatcttcctcaaggataggtgatagctcacagggagacaacaatttgcaaaaattaggccctgatgatgtgtctgtggatattgatgccattagaagggtctacaccagattgctctctaatgaaaaaattgaaactgcctttctcaatgcacttgtatatttgtcacctaacgtggaatgtgacttgacgtatcacaatgtatactctcgagatcctaattatctgaatttgttcattatcgtaatggagaatagaaatctccacagtcctgaatatctggaaatggctttgccattattttgcaaagcgatgagcaagctaccccttgcagcccaaggaaaactgatcagactgtggtctaaatacaatgcagaccagattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcaggaacttttgggagaagaaagaagaaacaagaaaggtcctcgagtggaccccctggaaactgaacttggtgttaaaaccctggattgtcgaaaaccacttatcccttttgaagagtttattaatgaaccactgaatgaggttctagaaatggataaagattatacttttttcaaagtagaaacagagaacaaattctcttttatgacatgtccctttatattgaatgctgtcacaaagaatttgggattatattatgacaatagaattcgcatgtacagtgaacgaagaatcactgttctctacagcttagttcaaggacagcagttgaatccatatttgagactcaaagttagacgtgaccatatcatagatgatgcacttgtccggctagagatgatcgctatggaaaatcctgcagacttgaagaagcagttgtatgtggaatttgaaggagaacaaggagttgatgagggaggtgtttccaaagaattttttcagctggttgtggaggaaatcttcaatccagatattggtatgttcacatacgatgaatctacaaaattgttttggtttaatccatcttcttttgaaactgagggtcagtttactctgattggcatagtactgggtctggctatttacaataactgtatactggatgtacattttcccatggttgtctacaggaagctaatggggaaaaaaggaacttttcgtgacttgggagactctcacccagttctatatcagagtttaaaagatttattggagtatgaagggaatgtggaagatgacatgatgatcactttccagatatcacagacagatctttttggtaacccaatgatgtatgatctaaaggaaaatggtgataaaattccaattacaaatgaaaacaggaaggaatttgtcaatctttattctgactacattctcaataaatcagtagaaaaacagttcaaggcttttcggagaggttttcatatggtgaccaatgaatctcccttaaagtacttattcagaccagaagaaattgaattgcttatatgtggaagccggaatctagatttccaagcactagaagaaactacagaatatgacggtggctataccagggactctgttctgattagggagttctgggaaatcgttcattcatttacagatgaacagaaaagactcttcttgcagtttacaacgggcacagacagagcacctgtgggaggactaggaaaattaaagatgattatagccaaaaatggcccagacacagaaaggttacctacatctcatacttgctttaatgtgcttttacttccggaatactcaagcaaagaaaaacttaaagagagattgttgaaggccatcacgtatgccaaaggatttggcatgctgtaaaacaaaacaaaacaaaataaaacaaaaaaaaggaagg

SEQ ID NO: 18 - protein, 24 aa, Homo sapiens
MALWMRLLPLLALLALWGPDPAAA

SEQ ID NO: 19 - protein, 20 aa, Homo sapiens
METDTLLLWVLLLWVPGSTG

SEQ ID NO: 20 - protein, 16 aa, Homo sapiens
RQIKIWFQNRRMKWKK

SEQ ID NO: 21 - protein, 9 aa, Homo sapiens
RRWWRRWRR

SEQ ID NO: 22 - protein, 18 aa, Homo sapiens
LLIILRRRIRKQAHAHSK

(19) United States
(12) Patent Application Publication
(10) Pub. No.: US 2020/0113955 A1
(43) Pub. Date: Apr. 16, 2020

(54) MODIFIED UBE3A GENE FOR A GENE THERAPY APPROACH FOR ANGELMAN SYNDROME

(71) Applicants: Kevin Ron Nash, Temple Terrace, FL (US); Edwin John Weeber, Apollo Beach, FL (US)

(72) Inventors: Kevin Ron Nash, Temple Terrace, FL (US); Edwin John Weeber, Apollo Beach, FL (US)

(73) Assignee: University of South Florida, Tampa, FL (US)

(21) Appl. No.: 16/716,785

(22) Filed: Dec. 17, 2019

Related U.S. Application Data
(63) Continuation of application No. PCT/US2018/039980, filed on Jun. 28, 2018.
(60) Provisional application No. 62/525,787, filed on Jun. 28, 2017.

(51) Int. Cl. A61K 35/761; A61K 48/00; A61P 25/28; C12N 15/86; C07K 14/62

(52) U.S. Cl. CPC A61K 35/761; A61K 48/0058; A61K 48/0066; A61P 25/28; C12N 2810/854; C07K 14/62; C12N 2840/007; C12N 2810/40; C12N 15/86

(57) ABSTRACT

A novel vector, composition and method of treating a neurological disorder characterized by deficient UBE3A is presented. The UBE3A gene, which encodes for E6-AP, a ubiquitin ligase, was found to be responsible for Angelman syndrome (AS). A unique feature of this gene is that it undergoes maternal imprinting in a neuron-specific manner. In the majority of AS cases, there is a mutation or deletion in the maternally inherited UBE3A gene, although other cases are the result of uniparental disomy or mismethylation of the maternal gene. A UBE3A protein construct was generated with additional sequences that allow the secretion from cells and uptake by neighboring neuronal cells. This UBE3A vector may be used in gene therapy to confer a functional E6-AP protein into the neurons and rescue disease pathology.

### CROSS REFERENCE TO RELATED APPLICATIONS

This application is a continuation of and claims priority to International Patent Application No. PCT/US2018/039980, entitled “Modified UBE3A Gene for a Gene Therapy Approach for Angelman Syndrome”, filed Jun. 28, 2018 which claims priority to U.S. Provisional Patent Application Ser. No. 62/525,787, entitled “Modified UBE3A Gene for a Gene Therapy Approach for Angelman Syndrome”, filed Jun. 28, 2017, the contents of each of which are hereby incorporated by reference into this disclosure.

### FIELD OF INVENTION

This invention relates to treatment of Angelman syndrome. More specifically, the present invention provides therapeutic methods and compositions for treating Angelman syndrome.

### BACKGROUND OF THE INVENTION

Angelman syndrome (AS) is a genetic disorder affecting neurons, estimated to effect about one in every 15,000 births (Clayton-Smith, Clinical research on Angelman syndrome in the United Kingdom: observations on 82 affected individuals. Am J Med Genet. 1993 Apr. 1; 46(1):12-5), though the actual number of diagnosed AS cases is greater likely due to misdiagnosis.

Angelman syndrome is a continuum of impairment, which presents with delayed and reduced intellectual and developmental advancement, most notably regarding language and motor skills. In particular, AS is defined by little or no verbal communication, with some non-verbal communication, ataxia, and disposition that includes frequent laughing and smiling and excitable movement.

More advanced cases result in severe mental retardation, seizures that may be difficult to control that typically begin before or by three years of age, frequent laughter (Nicholls, New insights reveal complex mechanisms involved in genomic imprinting. Am J Hum Genet. 1994 May; 54(5):733-40), miroencephaly, and abnormal EEG. In severe cases, patients may not develop language or may only have use of 5-10 words. Movement is commonly jerky, and walking commonly is associated with hand flapping and a stiff-gait. The patients are commonly epileptic, especially earlier in life, and suffer from sleep apnea, commonly only sleeping for 5 hours at a time. They are social and desire human contact. In some cases, skin and eyes may have little or no pigment, they may possess sucking and swallowing problems, sensitivity to heat, and a fixation to water bodies. Studies in UBE3A-deficient mice show disturbances in long-term synaptic plasticity. There are currently no cures for Angelman syndrome, and treatment is palliative. For example, anticonvulsant medication is used to reduce epileptic seizures, and speech and physical therapy are used to improve language and motor skills.

The gene UBE3A is responsible for AS and it is unique in that it is one of a small family of human imprinted genes. UBE3A, found on chromosome 15, encodes for the homologous to E6AP C terminus (HECT) protein (E6-associated protein (E6AP) (Kishino, et al., UBE3A/E6-AP mutations cause Angelman syndrome. Nat Gen. 1997 Jan. 15.15(1):70-3). UBE3A undergoes spatially-defined maternal imprinting in the brain; thus, the paternal copy is silenced via DNA methylation (Albrecht, et al., Imprinted expression of the murine Angelman syndrome gene, Ube3a, in hippocampal and Purkinje neurons. Nat Genet. 1997 September; 17(1):75-8). As such, only the maternal copy is active, the paternal chromosome having little or no effect on the proteosome of the neurons in that region of the brain. Inactivation, translocation, or deletion of portions of chromosome 15 therefore results in uncompensated loss of function. Some studies suggest improper E3-AP protein levels alter neurite contact in Angelman syndrome patients (Tonazzini, et al., Impaired neurite contract guidance in ubuitin ligase E3a (Ube3a)-deficient hippocampal neurons on nanostructured substrates. Adv Healthc Mater. 2016 April; 5(7):850-62).

The majority of Angelman's syndrome cases (70%) occur through a de novo deletion of around 4 Mb from 15q11-q13 of the maternal chromosome which incorporates the UBE3A gene (Kaplan, et al., Clinical heterogeneity associated with deletions in the long arm of chromosome 15: report of 3 new cases and their possible significance. Am J Med Genet. 1987 September; 28(1):45-53), but it can also occur as a result of abnormal methylation of the maternal copy, preventing its expression (Buiting, et al., Inherited microdeletions in the Angelman and Prader-Willi syndromes define an imprinting centre on human chromosome 15. Nat Genet. 1995 April; 9(4):395-400; Gabriel, et al., A transgene insertion creating a heritable chromosome deletion mouse model of Prader-Willi and Angelman syndrome. Proc Natl Acad Sci U.S.A. 1999 August; 96(16):9258-63) or uniparental disomy in which two copies of the paternal gene are inherited (Knoll, et al., Angelman and Prader-Willi syndromes share a common chromosome 15 deletion but differ in parental origin of the deletion. Am J Med Genet. 1989 Fed; 32(2):285-90; Malcolm, et al., Uniparental paternal disomy in Angelman's syndrome. Lancet. 1991 Mar. 23; 337(8743):694-7). The remaining AS cases arise through various UBE3A mutations of the maternal chromosome or they are diagnosed without a genetic cause (12-15UBE3A codes for the E6-associated protein (E6-AP) ubiquitin ligase. E6-AP is an E3 ubiquitin ligase, therefore it exhibits specificity for its protein targets, which include the tumor suppressor molecule p53 (Huibregtse, et al., A cellular protein mediates association of p53 with the E6 oncoprotein of human papillomavirus types 16 or 18. EMBO J. 1991 December; 10(13):4129-35), a human homologue to the yeast DNA repair protein Rad23 (Kumar, et al., Identification of HHR23A as a substrate for E6-associated protein-mediated ubiquitination. J Biol Chem. 1999 Jun. 25; 274(26):18785-92), E6-AP itself, and Arc, the most recently identified target (Nuber, et al., The ubiquitin-protein ligase E6-associated protein (E6-AP) serves as its own substrate. Eur J Biochem. 1998 Jun. 15; 254(3):643-9; Greer, et al., The Angelman Syndrome protein Ube3A regulates synapse Development by ubiquitinating arc. Cell. 2010 Mar. 5; 140(5): 704-16).

Mild cases are likely due to a mutation in the UBE3A gene at chromosome 15q11-13, which encodes for E6-AP ubiquitin ligase protein of the ubiquitin pathway, and more severe cases resulting from larger deletions of chromosome 15. Commonly, the loss of the UBE3A gene in the hippocampus and cerebellum result in Angelman syndrome, though single loss-of-function mutations can also result in the disorder.

The anatomy of the mouse and human AS brain shows no major alterations compared to the normal brain, indicating the cognitive deficits may be biochemical in nature as opposed to developmental (Jiang, et al., Mutation of the Angelman ubiquitin ligase in mice causes increased cytoplasmic p53 and deficits of contextual learning and long-term potentiation. Neuron. 1998 October; 21(4):799-811; Davies, et al., Imprinted gene expression in the brain. Neurosci Biobehav Rev. 2005 May; 29(3):421-430). An Angelman syndrome mouse model possessing a disruption of the maternal UBE3A gene through a null mutation of exon 2 (Jiang, et al., Mutation of the Angelman ubiquitin ligase in mice causes increased cytoplasmic p53 and deficits of contextual learning and long-term potentiation. Neuron. 1998 October; 21(4):799-811) was used. This model has been incredibly beneficial to the field of AS research due to its ability in recapitulating the major phenotypes characteristic of AS patients. For example, the AS mouse has inducible seizures, poor motor coordination, hippocampal-dependent learning deficits, and defects in hippocampal LTP. Cognitive deficits in the AS mouse model were previously shown to be associated with abnormalities in the phosphorylation state of calcium/calmodulin-dependent protein kinase II (CaMKII) (Weeber, et al., Derangements of hippocampal calcium/calmodulin-dependent protein kinase II in a mouse model for Angelman mental retardation syndrome. J Neurosci. 2003 April; 23(7):2634-44). There was a significant increase in phosphorylation at both the activating Thr²⁸⁶ site as well as the inhibitory Thr³⁰⁵ site of αCaMKII without any changes in total enzyme level, resulting in an overall decrease in its activity. There was also a reduction in the total amount of CaMKII at the postsynaptic density, indicating a reduction in the amount of active CaMKII. Crossing a mutant mouse model having a point mutation at the Thr³⁰⁵ site preventing phosphorylation with the AS mouse rescued the AS phenotype. i.e. seizure activity, motor coordination, hippocampal-dependent learning, and LTP were restored similar to wildtype levels. Thus, postnatal expression of αCaMKII suggests that the major phenotypes of the AS mouse model are due to postnatal biochemical alterations as opposed to a global developmental defect (Bayer, et al., Developmental expression of the CaM kinase II isoforms: ubiquitous γ- and δ-CaM kinase II are the early isoforms and most abundant in the developing nervous system. Brain Res Mol Brain Res. 1999 Jun. 18; 70(1):147-54).

Deficiencies in Ube3a are also linked in Huntington's disease (Maheshwari, et al., Deficiency of Ube3a in Huntington's disease mice brain increases aggregate load and accelerates disease pathology. Hum Mol Genet. 2014 Dec. 1; 23(23):6235-45).

Matentzoglu noted E6-AP possesses non-E3 activity related to hormone signaling (Matentzoglu, EP 2,724,721 A1). As such, administration of steroids, such as androgens, estrogens, and glucocorticoids, was used for treating various E6-AP disorders, including Angelman syndrome, autism, epilepsy, Prader-Willi syndrome, cervical cancer, fragile X syndrome, and Rett syndrome. Philpot suggested using a topoisomerase inhibitor to demethylate silenced genes thereby correcting for deficiencies in Ube3A (Philpot, et al., P.G. Pub. US 2013/0317018 A1). However, work in the field, and proposed therapeutics, do not address the underlying disorder, as in the use of steroids, or may result in other disorders, such as autism, where demethylation compounds are used. Accordingly, what is needed is a therapeutic that addresses the underlying cause of UBE3A deficiency disorders, in a safe, efficacious manner.

Nash & Weeber (WO 2016/179584) demonstrated that recombinant adeno-associated virus (rAAV) vectors can be an effective method for gene delivery in mouse models. However, only a small population of neurons are successfully transduced and thus express the protein, preventing global distribution of the protein in the brain as needed for efficacious therapy. As such, what is needed is a therapeutic that provides for supplementation of Ube3a protein throughout the entire brain.

### SUMMARY OF THE INVENTION

While most human disorders characterized by severe mental retardation involve abnormalities in brain structure, no gross anatomical changes are associated with AS. A Ube3a protein has been generated containing an appended to a cellular secretion sequence that allows the secretion of Ube3a from cells and cellular uptake sequence that provides uptake by neighboring neuronal cells. This provides a functional E6-AP protein into the neurons thereby rescuing from disease pathology.

The efficacy of novel plasmid constructs containing a modified Ube3A gene with secretion signals to promote E6-AP secretion and cell-penetrating peptide (CPP) signals to promote E6-AP reuptake in neighboring cells were examined. This allows for a greater global distribution of E6-AP upon transduction into a mouse brain, as a gene therapy for AS.

As such, a UBE3A vector was formed using a transcription initiation sequence, and a UBE construct disposed downstream of the transcription initiation sequence. The UBE construct is formed of a UBE3A sequence, a secretion sequence, and a cell uptake sequence. Nonlimiting examples of the UBE3A sequence include Mus musculus UBE3A, Homo sapiens UBE3A variant 1, variant 2, or variant 3. Nonlimiting examples of the cell uptake sequence include penetratin, R6W3, HIV TAT, HIV TATk and pVEC. Nonlimiting examples of the secretion sequence include insulin, GDNF and IgK.

In some variations of the invention, the transcription initiation sequence is a cytomegalovirus chicken-beta actin hybrid promoter, or human ubiquitin c promoter. The invention optionally includes an enhancer sequence. A nonlimiting example of the enhancer sequence is a cytomegalovirus immediate-early enhancer sequence disposed upstream of the transcription initiation sequence. The vector optionally also includes a woodchuck hepatitis post-transcriptional regulatory element.

In variations, the vector is inserted into a plasmid, such as a recombinant adeno-associated virus serotype 2-based plasmid. In specific variations, the recombinant adeno-associated virus serotype 2-based plasmid lacks DNA integration elements. A nonlimiting example of the recombinant adeno-associated virus serotype 2-based plasmid is a pTR plasmid.

In some variations, the secretion sequence is disposed upstream of the UBE3A sequence. The cell uptake sequence may be disposed upstream of the UBE3A sequence and downstream of the secretion sequence.

Also presented is a method of treating a neurodegenerative disorder characterized by UBE3A deficiency such as Angelman syndrome and Huntington's disease, by administering a therapeutically effective amount of UBE3A vector, as described previously, to the brain of a patient in order to correct the UBE3A deficiency. The vector may be administered by injection into the brain, such as by intrahippocampal or intraventricular injection. In some instances, the vector may be injected bilaterally. Exemplary dosages can range between about 5.55×10¹¹ to 2.86×10¹² genomes/g brain mass.

A composition for use in treating a neurodegenerative disorder characterized by UBE3A deficiency is also presented. The composition may be comprised of a UBE3A vector as described above, and a pharmaceutically acceptable carrier. In some instances, the pharmaceutically acceptable carrier can be a blood brain barrier permeabilizer such as mannitol.

### BRIEF DESCRIPTION OF THE DRAWINGS

For a fuller understanding of the invention, reference should be made to the following detailed description, taken in connection with the accompanying drawings, in which:

FIG. 1 is a dot blot of anti-GFP on media from HEK293 cells transfected with GFP clones containing signal peptides as indicated.

FIG. 2 is a map of the mouse UBE3A vector construct used in the present invention. Major genes are noted.

FIG. 3 is a Western blot showing secretion of E6-AP protein from plasmid transfected HEK293 cells. Culture media taken from control cells transfected cell culture media (cnt txn), media from Ube3a transfected cells (Ube3a txn); and media from untransfected cells (cnt untxn) were run on an acrylamide gel and anti-E6-AP antibody.

FIG. 4 is a graph of percentage area staining for E6-AP protein. Nontransgenic (Ntg) control mice shows the level of Ube3a expression in a normal mouse brain. Angelman syndrome mice (AS) show staining level in those mice (aka background staining). Injection of AAV4-STUb into the lateral ventricles of an AS mouse shows the level of E6-AP protein staining is increased as compared to an AS mouse. n=2.

FIG. 5 is a microscopic image of anti-E6-AP staining in a nontransgenic mouse. GFP (green fluorescent protein) is a cytosolic protein which is not secreted. This suggests that the Ube3a is being released from the ependymal cells and taken up in the parenchyma.

FIG. 6 is a microscopic image of anti-E6-AP staining in a nontransgenic mouse showing higher magnification images of the ventricular system (Lateral ventricle (LV), 3^{rd} ventricle). GFP (green fluorescent protein) is a cytosolic protein which is not secreted. This suggests that the Ube3a is being released from the ependymal cells and taken up in the parenchyma.

FIG. 7 is a microscopic image of anti-E6-AP staining in an uninjected AS mouse.

FIG. 8 is a microscopic image of anti-E6-AP staining in an uninjected AS mouse. showing higher magnification images of the ventricular system (Lateral ventricle (LV), 3^{rd} ventricle).

FIG. 9 is a microscopic image of anti-E6-AP staining in an AS mouse injected into the lateral ventricle with AAV4-STUb. Expression can be seen in the ependymal cells but staining is also observed in the parenchyma immediately adjacent to the ventricles (indicated with arrows). GFP (green fluorescent protein) is a cytosolic protein which is not secreted. This suggests that the Ube3a is being released from the ependymal cells and taken up in the parenchyma.

FIG. 10 is a microscopic image of anti-E6-AP staining in an AS mouse injected into the lateral ventricle with AAV4-STUb showing higher magnification images of the ventricular system (Lateral ventricle (LV), 3^{rd} ventricle). Expression can be seen in the ependymal cells but staining is also observed in the parenchyma immediately adjacent to the ventricles (indicated with arrows). GFP (green fluorescent protein) is a cytosolic protein which is not secreted. This suggests that the Ube3a is being released from the ependymal cells and taken up in the parenchyma.

FIG. 11 is a microscopic image of anti-E6-AP staining in an AS mouse injected into the lateral ventricle with AAV4-STUb. Higher magnification images of the ventricular system (Lateral ventricle (LV)) of Ube3a expression after AAV4-STUb delivery. Expression can be seen in the ependymal cells but staining is also observed in the parenchyma immediately adjacent to the ventricles (indicated with arrows). GFP (green fluorescent protein) is a cytosolic protein which is not secreted. This suggests that the Ube3a is being released from the ependymal cells and taken up in the parenchyma.

FIG. 12 is a microscopic image of anti-E6-AP staining in an AS mouse injected into the lateral ventricle with AAV4-STUb. Higher magnification images of the ventricular system (3^{rd} ventricle) of Ube3a expression after AAV4-STUb delivery. Expression can be seen in the ependymal cells but staining is also observed in the parenchyma immediately adjacent to the ventricles (indicated with arrows). GFP (green fluorescent protein) is a cytosolic protein which is not secreted. This suggests that the Ube3a is being released from the ependymal cells and taken up in the parenchyma.

FIG. 13 is a microscopic image of anti-E6-AP staining in a nontransgenic mouse transfected with GFP. Expression is not observed with the AAV4-GFP injections, which shows only transduction of the ependymal and choroid plexus cells. GFP (green fluorescent protein) is a cytosolic protein which is not secreted. This suggests that the Ube3a is being released from the ependymal cells and taken up in the parenchyma.

FIG. 14 is a microscopic image of anti-E6-AP staining in an AS mouse injected into the lateral ventricle with AAV4-STUb. Sagittal cross section of the brain of Ube3a expression after AAV4-STUb delivery.

FIG. 15 is a microscopic image of anti-E6-AP staining in an AS mouse injected into the lateral ventricle with AAV4-STUb. Sagittal cross section of the lateral ventricle (LV) in the brain showing Ube3a expression after AAV4-STUb delivery.

FIG. 16 is a microscopic image of anti-E6-AP staining in an AS mouse injected into the lateral ventricle with AAV4-STUb. Sagittal cross section of the 3^{rd} ventricle (3V) in the brain showing Ube3a expression after AAV4-STUb delivery.

FIG. 17 is a microscopic image of anti-E6-AP staining in an AS mouse injected into the lateral ventricle with AAV4-STUb. Sagittal cross section of the interior horn of the lateral ventricle (LV) in the brain showing Ube3a expression after AAV4-STUb delivery.

FIG. 18 is a microscopic image of anti-E6-AP staining in an AS mouse injected into the lateral ventricle with AAV4-STUb. Sagittal cross section of the lateral ventricle (4V) in the brain showing Ube3a expression after AAV4-STUb delivery.

FIG. 19 is a microscopic image of anti-E6-AP staining in an AS mouse injected into the lateral ventricle with AAV4-STUb. Sagittal cross section of the fourth ventricle (LV) in the brain showing Ube3a expression after AAV4-STUb delivery.

FIG. 20 is a microscopic image of anti-E6-AP staining in an AS mouse injected into the lateral ventricle with AAV4-STUb. Sagittal cross section of the brain with higher magnification images of the ventricular system on the lateral ventricle (LV), and (C) 3^{rd} ventricle (3V) of Ube3a expression after AAV4-STUb delivery.

FIG. 21 is a map of the human UBE3A vector construct used in the present invention. Major genes are noted.

FIG. 22 is a Western blot of HEK293 cell lysate transfected with hSTUb construct. The proteins were stained with anti-E6AP.

FIG. 23 is a dot blot with Anti-E6AP of HEK293 cells transfected with hSTUb construct with GDNF signal or insulin signal, shows insulin signal works better for expression and secretion.

FIG. 24 is a dot blot confirming insulin signal secretion using anti-HA tag antibody.

FIG. 25(A) is an illustration of the plasmid construct f for the GFP protein.

FIG. 25(B) is an image of gel electrophoresis result for the GFP protein.

FIG. 25(C) is a dot blot for different secretion signals using the GFP construct. The construct with the secretion signal was transduced into cell cultures and two clones obtained from each. The clones were cultured and media collected.

FIG. 26(A) is an illustration of the plasmid construct f for the E6-AP protein.

FIG. 26(B) is an image of gel electrophoresis result for the E6-AP protein.

FIG. 26(C) is a dot blot for different secretion signals using the E6-AP construct. The construct with the secretion signal was transduced into cell cultures and two clones obtained from each. The clones were cultured and media collected.

FIG. 27 is a Western blot showing the efficacy of cellular peptide uptake signals in inducing reuptake of the protein by neurons in transfected HEK293 cells. The cell lyses were added to new cell cultures of HEK293 cells and the concentration of E6-AP in these cells after incubation measured via Western blot.

FIG. 28(A) is a graph showing field excitatory post-synaptic potentials. A construct of Ube3A version 1 (hUbev1), a secretion signal, and the CPP TATk was transduced via an rAAV vector into mouse models of AS. Long-term potentiation of the murine brain was measured via electrophysiology post-mortem and compared to GFP-transfected AS model control mice and wild-type control mice.

FIG. 28(B) is a graph showing field excitatory post-synaptic potentials. A construct of Ube3A version 1 (hUbev1), a secretion signal, and the CPP TATk was transduced via an rAAV vector into mouse models of AS. Long-term potentiation of the murine brain was measured via electrophysiology post-mortem and compared to GFP-transfected AS model control mice and wild-type control mice.

### DETAILED DESCRIPTION OF THE PREFERRED EMBODIMENT

As used herein, the singular forms “a,” “an” and “the” include plural referents unless the context clearly dictates otherwise. Thus, for example, reference to “a polypeptide” includes a mixture of two or more polypeptides and the like.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used in the specification and claims, the singular form “a”, “an” and “the” include plural references unless the context clearly dictates otherwise. For example, the term “a vector” includes a plurality of vectors.

As used herein, “about” means approximately or nearly and in the context of a numerical value or range set forth means ±15% of the numerical.

“Adeno-associated virus (AAV) vector” as used herein refers to an adeno-associated virus vector that can be engineered for specific functionality in gene therapy. In some instances, the AAV can be a recombinant adeno-associated virus vector, denoted rAAV. While AAV4 is described for use herein, any suitable AAV known in the art can be used, including, but not limited to, AAV9, AAV5, AAV1 and AAV4.

“Administration” or “administering” is used to describe the process in which compounds of the present invention, alone or in combination with other compounds, are delivered to a patient. The composition may be administered in various ways including injection into the central nervous system including the brain, including but not limited to, intrastriatal, intrahippocampal, ventral tegmental area (VTA) injection, intracerebral, intracerebellar, intramedullary, intranigral, intraventricular, intracisternal, intracranial, intraparenchymal including spinal cord and brain stem; oral; parenteral (referring to intravenous and intraarterial and other appropriate parenteral routes); intrathecal; intramuscular; subcutaneous; rectal; and nasal, among others. Each of these conditions may be readily treated using other administration routes of compounds of the present invention to treat a disease or condition.

“Treatment” or “treating” as used herein refers to any of: the alleviation, amelioration, elimination and/or stabilization of a symptom, as well as delay in progression of a symptom of a particular disorder. For example, “treatment” of a neurodegenerative disease may include any one or more of the following: amelioration and/or elimination of one or more symptoms associated with the neurodegenerative disease, reduction of one or more symptoms of the neurodegenerative disease, stabilization of symptoms of the neurodegenerative disease, and delay in progression of one or more symptoms of the neurodegenerative disease.

“Prevention” or “preventing” as used herein refers to any of: halting the effects of the neurodegenerative disease, reducing the effects of the neurodegenerative disease, reducing the incidence of the neurodegenerative disease, reducing the development of the neurodegenerative disease, delaying the onset of symptoms of the neurodegenerative disease, increasing the time to onset of symptoms of the neurodegenerative disease, and reducing the risk of development of the neurodegenerative disease.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. In some embodiments, the pharmaceutically acceptable carrier can be a blood brain permeabilizer including, but not limited to, mannitol. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention.

As used herein “animal” means a multicellular, eukaryotic organism classified in the kingdom Animalia or Metazoa. The term includes, but is not limited to, mammals. Nonlimiting examples include rodents, mammals, aquatic mammals, domestic animals such as dogs and cats, farm animals such as sheep, pigs, cows and horses, and humans. Wherein the terms “animal” or the plural “animals” are used, it is contemplated that it also applies to any animals.

As used herein the phrase “conservative substitution” refers to substitution of amino acids with other amino acids having similar properties (e.g. acidic, basic, positively or negatively charged, polar or non-polar). The following six groups each contain amino acids that are conservative substitutions for one another: 1) alanine (A), serine (S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W).

As used herein “conservative mutation”, refers to a substitution of a nucleotide for one which results in no alteration in the encoding for an amino acid, i.e. a change to a redundant sequence in the degenerate codons, or a substitution that results in a conservative substitution. An example of codon redundancy is seen in Tables 1 and 2.

TABLE 1
Amino Acids (Category-Based) and
Triplet Code and Redundant
Corresponding Encoded Amino Acids
(Functional Group Category-Based)

| | | | |
|---|---|---|---|
| Nonpolar, aliphatic | | | |
| | Gly | G | GGT |
| | | | GGC |
| | | | GGA |
| | | | GGG |
| | Ala | A | GCT |
| | | | GCC |
| | | | GCA |
| | | | GCG |
| | Val | V | GTT |
| | | | GTC |
| | | | GTA |
| | | | GTG |
| | Leu | L | TTA |
| | | | TTG |
| | | | CTT |
| | | | CTC |
| | | | CTA |
| | | | CTG |
| | Met | M | ATG |
| | Ile | I | ATT |
| | | | ATC |
| | | | ATA |
| Aromatic | | | |
| | Phe | F | TTT |
| | | | TTC |
| | Tyr | Y | TAT |
| | | | TAC |
| | Trp | W | TGG |
| Negative charge | | | |
| | Asp | D | GAT |
| | | | GAC |
| | Glu | E | GAA |
| | | | GAG |
| Polar, uncharged | | | |
| | Ser | S | AGT |
| | | | AGC |
| | | | TCT |
| | | | TCC |
| | | | TCA |
| | | | TCG |
| | Thr | T | ACT |
| | | | ACC |
| | | | ACA |
| | | | ACG |
| | Cys | C | TGT |
| | | | TGC |
| | Pro | P | CCT |
| | | | CCC |
| | | | CCA |
| | | | CCG |
| | Asn | N | AAT |
| | | | AAC |
| | Gln | Q | CAA |
| | | | CAG |
| Positive charge | | | |
| | Lys | K | AAA |
| | | | AAG |
| | His | H | CAT |
| | | | CAC |
| | Arg | R | CGT |
| | | | CGC |
| | | | CGA |
| | | | CGG |
| | | | AGA |
| | | | AGG |
| OTHER | | | |
| | stop | | TTA |
| | | | TAG |
| | | | TGA |

TABLE 2
Redundant Triplet Code and Corresponding
Encoded Amino Acids.

| | U | | C | | A | | G | |
|---|---|---|---|---|---|---|---|---|
| U | UUU | Phe | UCU | Ser | UAU | Tyr | UGU | Cys |
| | UUC | Phe | UCC | Ser | UAC | Tyr | UGC | Cys |
| | UUA | Leu | UCA | Ser | UAA | END | UGA | END |
| | UUG | Leu | UCG | Ser | UAG | END | UGG | Trp |
| C | CUU | Leu | CCU | Pro | CAU | His | CGU | Arg |
| | CUC | Leu | CCC | Pro | CAC | His | CGC | Arg |
| | CUA | Leu | CCA | Pro | CAA | Gln | CGA | Arg |
| | CUG | Leu | CCG | Pro | CAG | Gln | CGG | Arg |
| A | AUU | Ile | ACU | Thr | AAU | Asn | AGU | Ser |
| | AUC | Ile | ACC | Thr | AAC | Asn | AGC | Ser |
| | AUA | Ile | ACA | Thr | AAA | Lys | AGA | Arg |
| | AUG | Met | ACG | The | AAG | Lys | AGG | Arg |
| G | GUU | Val | GCU | Ala | GAU | Asp | GGU | Gly |
| | GUC | Val | GCC | Ala | GAC | Asp | GGC | Gly |
| | GUA | Val | GCA | Ala | GAA | Glu | GGA | Gly |
| | GUG | Val | GCG | Ala | GAG | Glu | GGG | Gly |

Thus, according to Table 2, conservative mutations to the codon UUA include UUG, CUU, CUC, CUA, and CUG.

As used herein, the term “homologous” means a nucleotide sequence possessing at least 80% sequence identity, preferably at least 90% sequence identity, more preferably at least 95% sequence identity, and even more preferably at least 98% sequence identity to the target sequence. Variations in the nucleotide sequence can be conservative mutations in the nucleotide sequence, i.e. mutations in the triplet code that encode for the same amino acid as seen in the Table 2.

As used herein, the term “therapeutically effective amount” refers to that amount of a therapy (e.g., a therapeutic agent or vector) sufficient to result in the amelioration of Angelman syndrome or other UBE3A-related disorder or one or more symptoms thereof, prevent advancement of Angelman syndrome or other UBE3A-related disorder, or cause regression of Angelman syndrome or other UBE3A-related disorder. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

The dosing of compounds and compositions of the present invention to obtain a therapeutic or prophylactic effect is determined by the circumstances of the patient, as known in the art. The dosing of a patient herein may be accomplished through individual or unit doses of the compounds or compositions herein or by a combined or prepackaged or pre-formulated dose of a compounds or compositions. An average 40 g mouse has a brain weighing 0.416 g, and a 160 g mouse has a brain weighing 1.02 g, a 250 g mouse has a brain weighing 1.802 g. An average human brain weighs 1508 g, which can be used to direct the amount of therapeutic needed or useful to accomplish the treatment described herein.

Nonlimiting examples of dosages include, but are not limited to: 5.55×10¹¹ genomes/g brain mass, 5.75×10¹¹ genomes/g brain mass, 5.8×10¹¹ genomes/g brain mass, 5.9×10¹¹ genomes/g brain mass, 6.0×10¹¹ genomes/g brain mass, 6.1×10¹¹ genomes/g brain mass, 6.2×10¹¹ genomes/g brain mass, 6.3×10¹¹ genomes/g brain mass, 6.4×10¹¹ genomes/g brain mass, 6.5×10¹¹ genomes/g brain mass, 6.6.×10¹¹ genomes/g brain mass, 6.7×10¹¹ genomes/g brain mass, 6.8×10¹¹ genomes/g brain mass, 6.9.×10¹¹ genomes/g brain mass, 7.0×10¹¹ genomes/g brain mass, 7.1×10¹¹ genomes/g brain mass, 7.2×10¹¹ genomes/g brain mass, 7.3×10¹¹ genomes/g brain mass, 7.4×10¹¹ genomes/g brain mass, 7.5×10¹¹ genomes/g brain mass, 7.6×10¹¹ genomes/g brain mass, 7.7×10¹¹ genomes/g brain mass, 7.8×10¹¹ genomes/g brain mass, 7.9×10¹¹ genomes/g brain mass, 8.0×10¹¹ genomes/g brain mass, 8.1×10¹¹ genomes/g brain mass, 8.2×10¹¹ genomes/g brain mass, 8.3×10¹¹ genomes/g brain mass, 8.4×10¹¹ genomes/g brain mass, 8.5×10¹¹ genomes/g brain mass, 8.6×10¹¹ genomes/g brain mass, 8.7×10¹¹ genomes/g brain mass, 8.8×10¹¹ genomes/g brain mass, 8.9×10¹¹ genomes/g brain mass, 9.0×10¹¹ genomes/g brain mass, 9.1×10¹¹ genomes/g brain mass, 9.2×10¹¹ genomes/g brain mass, 9.3×10¹¹ genomes/g brain mass, 9.4×10¹¹ genomes/g brain mass, 9.5×10¹¹ genomes/g brain mass, 9.6×10¹¹ genomes/g brain mass, 9.7×10¹¹ genomes/g brain mass, 9.80×10¹¹ genomes/g brain mass, 1.0×10¹² genomes/g brain mass, 1.1×10¹² genomes/g brain mass, 1.2×10¹² genomes/g brain mass, 1.3×10¹² genomes/g brain mass, 1.4×10¹² genomes/g brain mass, 1.5×10¹² genomes/g brain mass, 1.6×10¹² genomes/g brain mass, 1.7×10¹² genomes/g brain mass, 1.8×10¹² genomes/g brain mass, 1.9×10¹² genomes/g brain mass, 2.0×10¹² genomes/g brain mass, 2.1×10¹² genomes/g brain mass, 2.2×10¹² genomes/g brain mass, 2.3×10¹² genomes/g brain mass, 2.40×10¹² genomes/g brain mass, 2.5×10¹² genomes/g brain mass, 2.6×10¹² genomes/g brain mass, 2.7×10¹² genomes/g brain mass, 2.75×10¹² genomes/g brain mass, 2.8×10¹² genomes/g brain mass, or 2.86×10¹² genomes/g brain mass.

The compositions used in the present invention may be administered individually, or in combination with or concurrently with one or more other therapeutics for neurodegenerative disorders, specifically UBE3A deficient disorders.

As used herein “patient” is used to describe an animal, preferably a human, to whom treatment is administered, including prophylactic treatment with the compositions of the present invention.

“Neurodegenerative disorder” or “neurodegenerative disease” as used herein refers to any abnormal physical or mental behavior or experience where the death or dysfunction of neuronal cells is involved in the etiology of the disorder. Further, the term “neurodegenerative disease” as used herein describes “neurodegenerative diseases” which are associated with UBE3A deficiencies. Exemplary neurodegenerative diseases include Angelman's Syndrome, Huntington's disease, Alzheimer's disease, Parkinson's disease, amyotrophic lateral sclerosis, autistic spectrum disorders, epilepsy, multiple sclerosis, Prader-Willi syndrome, Fragile X syndrome, Rett syndrome and Pick's Disease.

“UBE3A deficiency” as used herein refers to a mutation or deletion in the UBE3A gene.

The term “normal” or “control” as used herein refers to a sample or cells or patient which are assessed as not having Angelman syndrome or any other neurodegenerative disease or any other UBE3A deficient neurological disorder.

Generally, a UBE3A vector was formed using a transcription initiation sequence, and a UBE construct disposed downstream of the transcription initiation sequence. The UBE construct is formed of a UBE3A sequence, a secretion sequence, and a cell uptake sequence. Nonlimiting examples of the UBE3A sequence are SEQ ID No: 4, SEQ ID No: 9, SEQ ID No: 14, SEQ ID No:15, SEQ ID NO: 17, a cDNA of SEQ ID No: 10, a cDNA of SEQ ID No: 16, or a homologous sequence. Variations of the DNA sequence include conservative mutations in the DNA triplet code, as seen in Tables 1 and 2. In specific variations, the UBE3A sequence is Mus musculus UBE3A, Homo sapiens UBE3A variant 1, variant 2, or variant 3.

Nonlimiting examples of the secretion sequence are SEQ ID No: 2, SEQ ID No: 5, SEQ ID No: 11, SEQ ID No: 12, a cDNA of SEQ ID No: 3, a cDNA of SEQ ID NO: 7, a cDNA of SEQ ID NO: 18. A cDNA of SEQ ID NO: 19, or a homologous sequence, with variations of the DNA sequence that include the aforementioned conservative mutations.

Nonlimiting examples of the cell uptake sequence are SEQ ID No: 6, a cDNA of SEQ ID No. 8, a cDNA of SEQ ID No: 13, a cDNA of SEQ ID No: 20, a cDNA of SEQ ID No: 21, a cDNA of SEQ ID No: 22, or a homologous sequence. Variations of the DNA sequence include the aforementioned conservative mutations.

In specific variations of the invention, the secretion sequence is disposed upstream of the UBE3A sequence, and more specifically is optionally is disposed upstream of the UBE3A sequence and downstream of the secretion sequence. Other possible uptake proteins include penetratin, TATk, pVEC, transportan, MPG, Pep-1, polyarginines, MAP, and R6W3.

In some variations of the invention, the transcription initiation sequence is a cytomegalovirus chicken-beta actin hybrid promoter, or human ubiquitin c promoter. The invention optionally includes an enhancer sequence. A nonlimiting example of the enhancer sequence is a cytomegalovirus immediate-early enhancer sequence disposed upstream of the transcription initiation sequence. The vector optionally also includes a woodchuck hepatitis post-transcriptional regulatory element. The listed promotors, enhancer sequence and post-transcriptional regulatory element are well known in the art. (Garg S. et al., The hybrid cytomegalovirus enhancer/chicken beta-actin promotor along with woodchuck hepatitis virus posttranscriptional regulatory element enhances the protective efficacy of DNA vaccines, J. Immunol., Jul. 1, 2004; 173(1):550-558; Higashimoto, T. et al., The woodchuck hepatitis virus post-transcriptional regulatory element reduces readthrough transcription from retroviral vectors, September 2007; 14(17): 1298-304; Cooper, A. R. et al., Rescue of splicing-mediated intron loss maximizes expression in lentiviral vectors containing the human ubiquitin C promoter, Nucleic Acids Res., January 2015; 43(1):682-90).

In variations, the vector is inserted into a plasmid, such as a recombinant adeno-associated virus serotype 2-based plasmid. In specific variations, the recombinant adeno-associated virus serotype 2-based plasmid lacks DNA integration elements. A nonlimiting example of the recombinant adeno-associated virus serotype 2-based plasmid is a pTR plasmid.

A method of synthesizing the UBE3A vector includes inserting a UBE3A construct into a backbone plasmid having a transcription initiation sequence. The TBE3A construct is formed of a UBE3A sequence, a secretion sequence, and a cell uptake sequence as described above. For example, Ube3a gene was cloned and fused in frame to the 3′ DNA sequence (N-terminus with two other peptide sequences), signal peptide and HIV TAT sequences, which were cloned into a recombinant adeno-associated viral vector for expression of the secreted E6-AP protein in the brain and spinal cord of AS patients. The UBE construct is optionally inserted by cleaving the backbone plasmid with at least one endonuclease, and the UBE3A construct ligated to the cleaved ends of the backbone plasmid.

The vector was then optionally inserted into an amplification host, possessing an antibiotic resistance gene, and subjected to an antibiotic selection corresponding to the antibiotic resistance gene. The amplification host was then expanded in a medium containing the antibiotic selection and the expanded amplification host collected. The vector was then isolated from the amplification host. In specific variations of the invention, the antibiotic resistance gene is an ampicillin resistance gene, with the corresponding antibiotic selection, ampicillin.

In a preferred embodiment, a UBE3A vector is formed from cDNA cloned from a Homo sapiens UBE3A gene to form the UBE3A, version 1 gene (SEQ ID No: 9) which is fused to a gene encoding a secretion signaling peptide, such as GDNF, insulin or IgK. In a preferred embodiment, GDNF is used. The construct is inserted into the hSTUb vector, under a CMV chicken-beta actin hybrid promoter (preferred) or a human ubiquitin c promoter. Woodchuck hepatitis post-transcriptional regulatory element (WPRE) is present to increase expression levels.

The UBE3A-seretion signal construct is then attached to a cellular uptake peptide (cell penetrating peptide or CPP) such as HIV TAT or HIV TATk (preferred). The human UBE3A vector is then transformed into an amplification host such as E. coli using the heat shock method described in Example 2. The transformed E. coli were expanded in broth containing ampicillin to select for the vector and collect large amounts of vector. Therapeutically effective doses of vector can then the administered to a patient as a gene therapy for treating Angelman syndrome or another neurological disorder having UBE3A deficiency. The vector may be administered via injection into the hippocampus or ventricles, in some cases, bilaterally. Dosages of the therapeutic can range between about 5.55×10¹¹ to 2.86×10¹² genomes/g brain mass.

### Example 1—Efficiency of the Secretion Signal

To test the efficacy of the secretion signal, GFP (SEQ ID No: 1) (XM 013480425.1) was cloned in frame with human insulin, GDNF (SEQ ID No: 2) (AB675653.1) or IgK signal peptides.

(SEQ ID No: 1)
ATGGCTCGTC TTTCTTTTGT TTCTCTTCTT TCTCTGTCAC
TGCTCTTCGG GCAGCAAGCA GTCAGAGCTC AGAATTACAC
CATGGTGAGC AAGGGCGAGG AGCTGTTCAC CGGGGTGGTG
CCCATCCTGG TCGAGCTGGA CGGCGACGTA AACGGCCACA
AGTTCAGCGT GTCCGGCGAG GGCGAGGGCG ATGCCACCTA
CGGCAAGGAC TGCCTGAAGT TCATCTGCAC CACCGGCAAG
CTGCCCGTGC CCTGGCCCAC CCTCGTGACC ACCTTCGGCT
ACGGCCTGAT GTGCTTCGCC CGCTACCCCG ACCACATGAA
GCAGCACGAC TTCTTCAAGT CCGCCATGCC CGAAGGCTAC
GTCCAGGAGC GCACCATCTT CTTCAAGGAC GACGGCAACT
ACAAGACCCG CGCCGAGGTG AAGTTCGAGG GCGACACCCT
GGTGAACCGC ATCGAGCTGA AGGGCATCGA CTTCAAGGAG
GACGGCAACA TCCTGGGGCA CAAGCTGGAG TACAACTACA
ACAGCCACAA CGTCTATATC ATGGCCGACA AGCAGAAGAA
CGGCATCAAG GTGAACTTCA AGATCCGCCA CAACATCGAG
GACGGCAGCG TGCAGCTCGC CGACCACTAC CAGCAGAACA
CCCCCATCGG CGACGGCCCC GTGCTGCTGC CCGACAACCA
CTACCTGAGC TACCAGTCCG CCCTGAGCAA AGACCCCAAC
GAGAAGCGCG ATCACATGGT CCTGCTGGAG TTCGTGACCG
CCGCCGGGAT CACTCTCGGC ATGGACGAGC TATACAAGTG
GGCGCGCCAC TCGAGACGAA TCACTAGTGA ATTCGCGGCC
GCCTGCAGGT CGAGGTTTGC AGCAGAGTAG,

fused with a secretion protein based on GDNF;

(SEQ ID No: 2)
ATGAAGTTATGGGATGTCGTGGCTGTCTGCCTGGTGCTGCTCCACACC
GCGTCCGCC
(XM 017009337.2), which encodes
(SEQ ID NO: 3)
MKLWDVVAVCLVLLHTASA
(AAC98782.1)

The construct was inserted into a pTR plasmid and transfected into HEK293 cells (American Type Culture Collection, Manassas, Va.). HEK293 cells were grown at 37° C. 5% CO₂ in Dulbecco's Modified Essential Medium (DMEM) with 10% FBS and 1% Pen/Strep and subcultured at 80% confluence.

The vector (2 μg/well in a 6-well plate) was transfected into the cells using PEI transfection method. The cells were subcultured at 0.5×10⁶ cells per well in a 6-well plate with DMEM medium two days before the transfection. Medium was replaced the night before transfection. Endotoxin-free dH₂O was heated to at around 80° C., and polyethylenimine (Sigma-Aldrich Co. LLC, St. Louis, Mo.) dissolved. The solution was cooled to around 25° C., and the solution neutralized using sodium hydroxide. AAV4-STUb vector or negative control (medium only) was added to serum-free DMEM at 2 μg to every 200 μL for each well transfected, and 9 μL of 1 μg/L polyethylenimine added to the mix for each well. The transfection mix was incubated at room temperature for 15 minutes, then added to each well of cells at 210 μL per well and incubated for 48 hours.

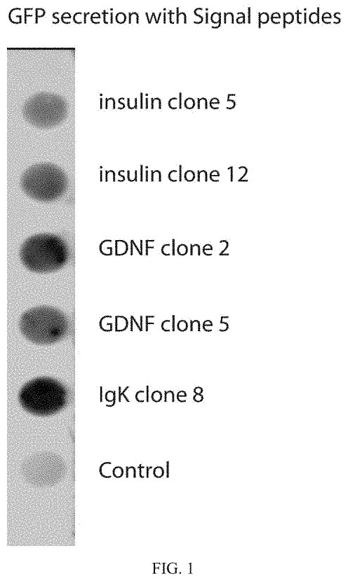

Media was collected from each culture well and 2 μL spotted onto a nitrocellulose membrane using a narrow-tipped pipette. After the samples dried, the membrane was blocked applying 5% BSA in TBS-T to the membrane and incubating at room temperature for 30 minutes to 1 hour, followed by incubating the membrane with chicken anti-GFP (5 μg/mL, Abcam PLC, Cambridge, UK; # ab13970) in BSA/TBS-T for 30 min at room temperature. The membrane was washed with TBS-T 3 times, 5 minutes for each wash. The membrane was incubated with anti-chicken HRP conjugate secondary antibody (Southern Biotechnology, Thermo Fisher Scientific, Inc., Waltham, Mass.; #6100-05, 1/3000) conjugated with HRP for 30 minutes at room temperature, followed by washing the membrane three times with TBS-T, once for 15 minutes, and subsequent washed at 5 minutes each. The membrane was washed with TBS for 5 minutes at room temperature, and incubated with luminescence reagent for 1 minute (Millipore, Merck KGaA, Darmstadt, DE; # WBKLS0100). The membrane was recorded on a GE Amersham Imager 600 (General Electric, Fairfield, Calif.), shown in FIG. 1.

As seen from FIG. 1, all three secretion signals resulted in release of GFP-tagged protein from cells as observed by comparison to untransfected control cells. Of the three secretion constructs, the IgK construct showed the highest level of secretion, though clone 2 of the GDNF construct did display similarly high secretion of GFP-tagged protein.

### Example 2—Mouse-UBE3A Vector Construct

A mouse-UBE3A vector construct was generated using a pTR plasmid. The mouse (Mus musculus) UBE3A gene was formed from cDNA (U82122.1);

(SEQ ID No: 4)
ATGAAGCGAG CAGCTGCAAA GCATCTAATA GAACGCTACT
ACCATCAGTT AACTGAGGGC TGTGGAAATG AGGCCTGCAC
GAATGAGTTT TGTGCTTCCT GTCCAACTTT TCTTCGTATG
GATAACAATG CAGCAGCTAT TAAAGCCCTT GAGCTTTATA
AAATTAATGC AAAACTCTGT GATCCTCATC CCTCCAAGAA
AGGAGCAAGC TCAGCTTACC TTGAGAACTC AAAAGGTGCA
TCTAACAACT CAGAGATAAA AATGAACAAG AAGGAAGGAA
AAGATTTTAA AGATGTGATT TACCTAACTG AAGAGAAAGT
ATATGAAATT TATGAATTTT GTAGAGAGAG TGAGGATTAT
TCCCCTTTAA TTCGTGTAAT TGGAAGAATA TTTTCTAGTG
CTGAGGCACT GGTTCTGAGC TTTCGGAAAG TCAAACAGCA
CACAAAGGAG GAATTGAAAT CTCTTCAAGA AAAGGATGAA
GACAAGGATG AAGATGAAAA GGAAAAAGCT GCATGTTCTG
CTGCTGCTAT GGAAGAAGAC TCAGAAGCAT CTTCTTCAAG
GATGGGTGAT AGTTCACAGG GAGACAACAA TGTACAAAAA
TTAGGTCCTG ATGATGTGAC TGTGGATATT GATGCTATTA
GAAGGGTCTA CAGCAGTTTG CTCGCTAATG AAAAATTAGA
AACTGCCTTC CTGAATGCAC TTGTATATCT GTCACCTAAC
GTGGAATGTG ATTTGACATA TCATAATGTG TATACTCGAG
ATCCTAATTA TCTCAATTTG TTCATTATTG TAATGGAGAA
TAGTAATCTC CACAGTCCTG AATATCTGGA AATGGCGTTG
CCATTATTTT GCAAAGCTAT GTGTAAGCTA CCCCTTGAAG
CTCAAGGAAA ACTGATTAGG CTGTGGTCTA AATACAGTGC
TGACCAGATT CGGAGAATGA TGGAAACATT TCAGCAACTT
ATTACCTACA AAGTCATAAG CAATGAATTT AATAGCCGAA
ATCTAGTGAA TGATGATGAT GCCATTGTTG CTGCTTCAAA
GTGTTTGAAA ATGGTTTACT ATGCAAATGT AGTGGGAGGG
GATGTGGACA CAAATCATAA TGAGGAAGAT GATGAAGAAC
CCATACCTGA GTCCAGCGAA TTAACACTTC AGGAGCTTCT
GGGAGATGAA AGAAGAAATA AGAAAGGTCC TCGAGTGGAT
CCACTAGAAA CCGAACTTGG CGTTAAAACT CTAGACTGTC
GAAAACCACT TATCTCCTTT GAAGAATTCA TTAATGAACC
ACTGAATGAT GTTCTAGAAA TGGACAAAGA TTATACCTTT
TTCAAAGTTG AAACAGAGAA CAAATTCTCT TTTATGACAT
GTCCCTTTAT ATTGAATGCT GTCACAAAGA ATCTGGGATT
ATATTATGAC AATAGAATTC GCATGTACAG TGAAAGAAGA
ATCACTGTTC TTTACAGCCT AGTTCAAGGA CAGCAGTTGA
ATCCGTATTT GAGACTCAAA GTCAGACGTG ACCATATTAT
AGATGATGCA CTGGTCCGGC TAGAGATGAT TGCTATGGAA
AATCCTGCAG ACTTGAAGAA GCAGTTGTAT GTGGAATTTG
AAGGAGAACA AGGAGTAATG AGGGAGGCGT TTCCAAAGAG
TTTTTTCAGT TGGGTTGTGG AGGAAATTTT TAATCCAAAT
ATTGGTATGT TCACATATGA TGAAGCTACG AAATTATTTT
GGTTTAATCC ATCTTCTTTT GAAACTGAGG GTCAGGTTTA
CTCTGATTGG CATATCCTGG GTCTGGCTAT TTACAATAAT
TGTATACTGG ATGTCCATTT TCCCATGGTT GTATACAGGA
AGCTAATGGG GAAAAAAGGA ACCTTTCGTG ACTTGGGAGA
CTCTCACCCA GTTTTATATC AGAGTTTAAA GGATTTATTG
GAATATGAAG GGAGTGTGGA AGATGATATG ATGATCACTT
TCCAGATATC ACAGACAGAT CTTTTTGGTA ACCCAATGAT
GTATGATCTA AAAGAAAATG GTGATAAAAT TCCAATTACA
AATGAAAACA GGAAGGAATT TGTCAATCTC TATTCAGACT
ACATTCTCAA TAAATCTGTA GAAAAACAAT TCAAGGCATT
TCGCAGAGGT TTTCATATGG TGACTAATGA ATCGCCCTTA
AAATACTTAT TCAGACCAGA AGAAATTGAA TTGCTTATAT
GTGGAAGCCG GAATCTAGAT TTCCAGGCAC TAGAAGAAAC
TACAGAGTAT GACGGTGGCT ATACGAGGGA ATCTGTTGTG
ATTAGGGAGT TCTGGGAAAT TGTTCATTCG TTTACAGATG
AACAGAAAAG ACTCTTTCTG CAGTTTACAA CAGGCACAGA
CAGAGCACCT GTTGGAGGAC TAGGAAAATT GAAGATGATT
ATAGCCAAAA ATGGCCCAGA CACAGAAAGG TTACCTACAT
CTCATACTTG CTTTAATGTC CTTTTACTTC CGGAATATTC
AAGCAAAGAA AAACTTAAAG AGAGATTGTT GAAGGCCATC
ACATATGCCA AAGGATTTGG CATGCTGTAA
(U82122.1).

The cDNA was subcloned and sequenced. The mouse UBE3A gene (SEQ ID No. 4) was fused to DNA sequences encoding the secretion signaling peptide GDNF (SEQ ID No. 5) and cell uptake peptide HIV TAT sequence (SEQ ID No: 6). The secretion signaling peptide has the DNA sequence;

(SEQ ID No: 5)
ATG GCC CTG TTG GTG CAC TTC CTA CCC CTG CTG GCC
CTG CTT GCC CTC TGG GAG CCC AAA CCC ACC CAG GCT
TTT GTC
(NM 008386.4), encoding to protein sequence;
(SEQ ID No: 7)
MALLVHFLPLLALLALWEPKPTQAFV
(NP 032412.3);

while HIV TAT sequence is;

(SEQ ID No: 6)
TAC GGC AGA AAG AAG AGG AGG CAG AGA AGG AGA,
encoding to protein sequence;
(SEQ ID No: 8)
YGRKKRRQRRR
(AIW51918.1).

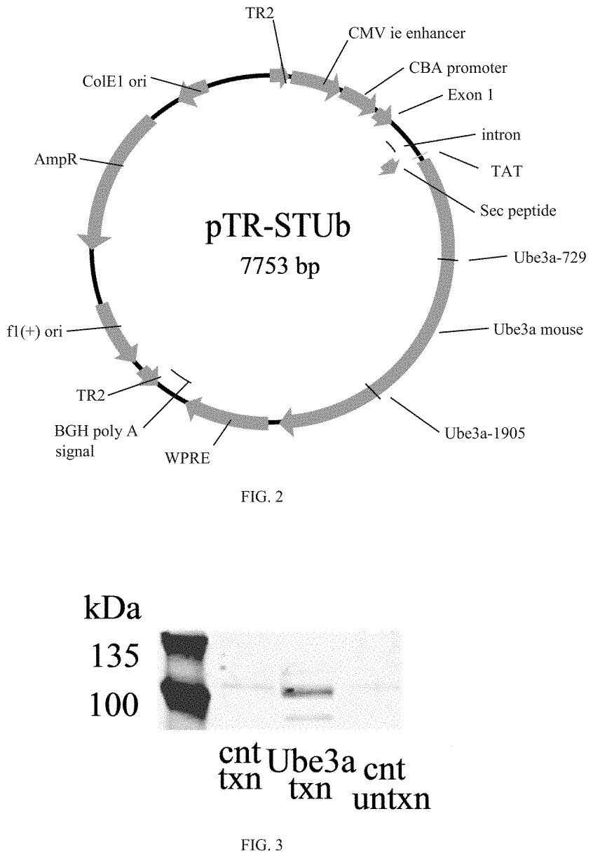

The construct sequence of SEQ ID No: 4 fused with SEQ ID No: 5 and SEQ ID No: 6 was inserted into a pTR plasmid. The plasmid was cleaved using Age I and Xho I endonucleases and the construct sequence ligated using ligase. The vector contains AAV serotype 2 terminal repeats, CMV-chicken-beta actin hybrid promoter and a WPRE, seen in FIG. 2. The recombinant plasmid lacks the Rep and Cap elements, limiting integration of the plasmid into host DNA.

The vector (AAV4-STUb vector) was then transformed into Escherichia coli (E. coli, Invitrogen, Thermo Fisher Scientific, Inc., Waltham, Mass.; SURE2 cells). Briefly, cells were equilibrated on ice and 1 pg to 500 ng of the vector were added to the E. coli and allowed to incubate for about 1 minute. The cells were electroporated with a BioRad Gene Pulser in a 0.1 cm cuvette (1.7V, 200 Ohms). The E. Coli were then grown in media for 60 min prior to being plated onto agar, such as ATCC medium 1065 (American Type Culture Collection, Manassas, Va.), with ampicillin (50 μg/mL). E. coli was expanded in broth containing ampicillin to collect large amounts of vector.

### Example 3—In Vitro Testing of Mouse-UBE3A Vector Construct

The mouse vector properties of the construct generated in Example 2 were tested in HEK293 cells (American Type Culture Collection, Manassas, Va.). HEK293 cells were grown at 37° C. 5% CO₂ in Dulbecco's Modified Essential Medium (DMEM) with 10% FBS and 1% Pen/Strep and subcultured at 80% confluence.

The vector (2 μg/well in a 6-well plate) was transfected into the cells using PEI transfection method. The cells were subcultured at 0.5×10⁶ cells per well in a 6-well plate with DMEM medium two days before the transfection. Medium was replaced the night before transfection. Endotoxin-free dH₂O was heated to at around 80° C., and polyethylenimine (Sigma-Aldrich Co. LLC, St. Louis, Mo.) dissolved. The solution was allowed to cool to around 25° C., and the solution neutralized using sodium hydroxide. AAV4-STUb vector or negative control (medium only) was added to serum-free DMEM at 2 μg to every 200 μl for each well transfected, and 9p of 1 μg/μl polyethylenimine added to the mix for each well. The transfection mix was incubated at room temperature for 15 minutes, then added to each well of cells at 210 μl per well and incubated for 48 hours.

Media was collected from AAV4-STUb vector transfected cells, medium-only transfected control cells, and untransfected control cells. The medium was run on Western blot and stained with rabbit anti-E6-AP antibody (A300-351A, Bethyl Labs, Montgomery, Tex.), which is reactive against human and mouse E6-AP, at 0.4 μg/ml. Secondary conjugation was performed with rabbit-conjugated horseradish peroxidase (Southern Biotechnology, Thermo Fisher Scientific, Inc., Waltham, Mass.). The results were determined densiometrically, and show the HEK293 cells transfected with AAV4-STUb secrete E6-AP protein into the medium, as seen in FIG. 3.

### Example 4—In Vivo Testing of Mouse-UBE3A Vector Construct

Transgenic mice were formed by crossbreeding mice having a deletion in the maternal UBE3A (Jiang, et al., Mutation of the Angelman ubiquitin ligase in mice causes increased cytoplasmic p53 and deficits of contextual learning and long-term potentiation. Neuron. 1998 October; 21(4):799-811; Gustin, et al., Tissue-specific variation of Ube3a protein expression in rodents and in a mouse model of Angelman syndrome. Neurobiol Dis. 2010 September; 39(3):283-91; Heck, et al., Analysis of cerebellar function in Ube3a-deficient mice reveals novel genotype-specific behaviors. Hum Mol Genet. 2008 Jul. 15; 17(14):2181-9) and GABARB3. Mice were housed in a 12-hour day-light cycle and fed food and water ad libitum. Three month old mice were treated with the vector.

Mice were anesthetized with isoflurane and placed in the stereotaxic apparatus (51725D Digital Just for Mice Stereotaxic Instrument, Stoelting, Wood Dale, Ill.). An incision was made sagittally over the middle of the cranium and the surrounding skin pushed back to enlarge the opening. The following coordinates were used to locate the left and right hippocampus: AP 22.7 mm, L 62.7 mm, and V 23.0 mm. Mice received bilateral intrahippocampal injections of either AAV4-STUb particles at a concentration of 1×10¹² genomes/mL (N=2) in 10 μL of 20% mannitol or vehicle (10 μL of 20% mannitol) using a 10 mL Hamilton syringe in each hemisphere. The wound was cleaned with saline and closed using Vetbond (NC9286393 Fisher Scientific, Pittsburgh, Pa.). Control animals included uninjected AS mice and littermate wild type mice (n=2). Mice recovered in a clean, empty cage on a warm heating pad and were then singly housed until sacrificed. The mice were monitored over the course of the experiment.

At day 30 after treatment, the mice were euthanized by injecting a commercial euthanasia solution, Somnasol®, (0.22 ml/kg) intraperitoneally. After euthanizing the animals, CSF was collected and the animals were perfused with PBS and the brain removed. The brain was fixed in 4% paraformaldehyde solution overnight prior to cryoprotection in sucrose solutions. Brains were sectioned at 25 m using a microtome.

Most recombinant adeno-associated virus vector studies inject the vector directly into the parenchymal, which typically results in limited cellular transduction (Li, et al., Intra-ventricular infusion of rAAV-1-EGFP resulted in transduction in multiple regions of adult rat brain: a comparative study with rAAV2 and rAAV5 vectors. Brain Res. 2006 Nov. 29; 1122(1):1-9). However, appending a secretion signaling sequence and TAT sequence to the Ube3A protein allows for secretion of the HECT protein (i.e., UBE3A) from transfected cells and uptake of the peptide by adjacent neurons, allowing injection into a discrete site to serve as a supply of protein for other sites throughout the brain.

Brains from sacrificed mice were sliced using a microtome and stained for E6-AP protein using anti-E6-AP antibody (A300-351A, Bethyl Labs, Montgomery, Tex.) with a biotinylated anti-rabbit secondary antibody (Vector Labs # AB-1000). Staining was completed with ABC (Vector Labs) and DAB reaction. Sections were mounted and scanned using Zeiss Axio Scan microscope. Percentage area staining was quantified using IAE-NearCYTE image analysis software (University of Pittsburgh Starzl Transplant Institute, Pittsburgh, Pa.).

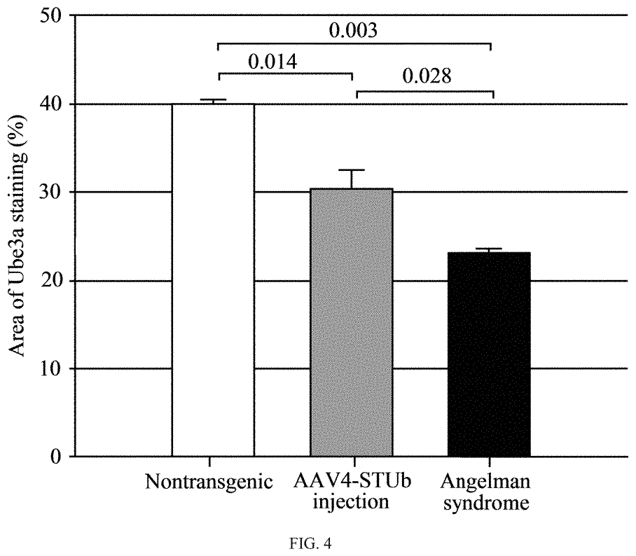

Nontransgenic (Ntg) control mice shows the level of UBE3a expression in a normal mouse brain, which was about 40%, as seen in FIG. 4. By comparison, Angelman syndrome mice (AS) show Ube3a protein staining levels of about 25%. Insertion of the AAV4-STUb vector into the lateral ventricles of an AS mouse shows the vector increased the level of E6-AP to around 30-35%.

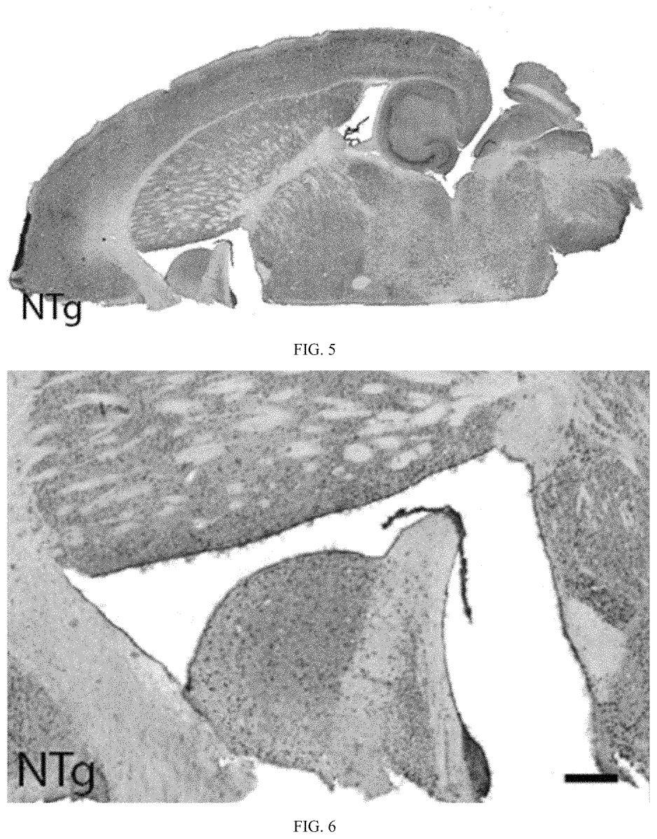

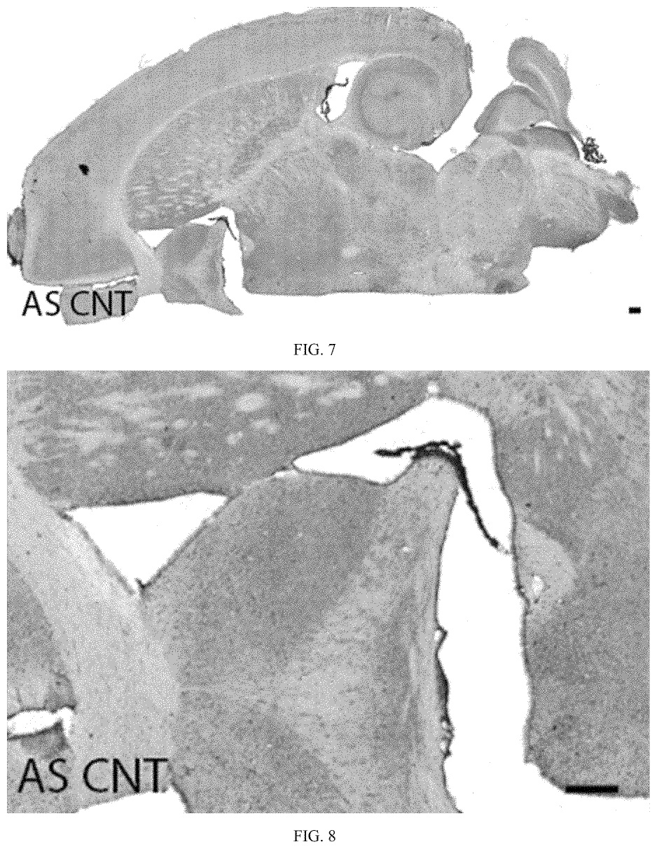

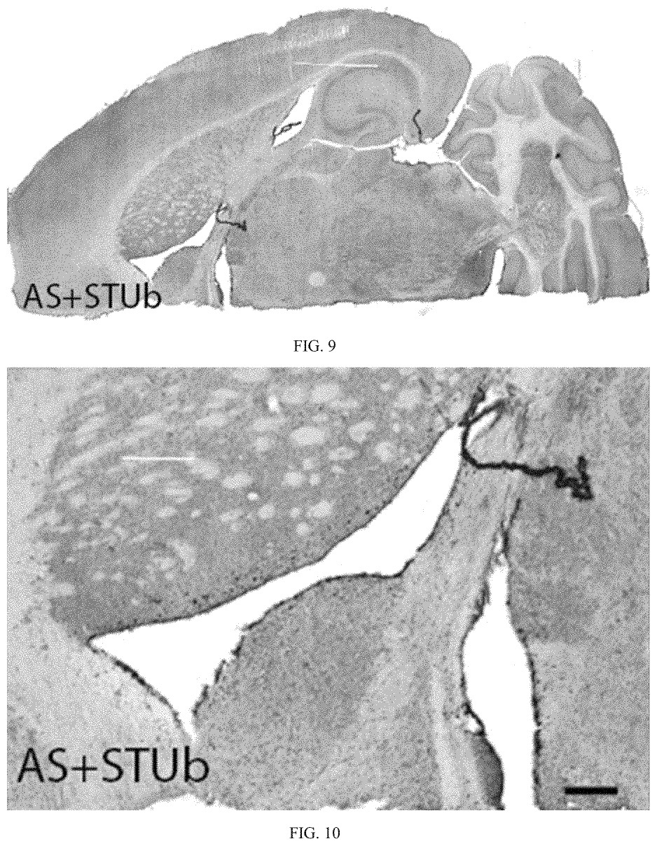

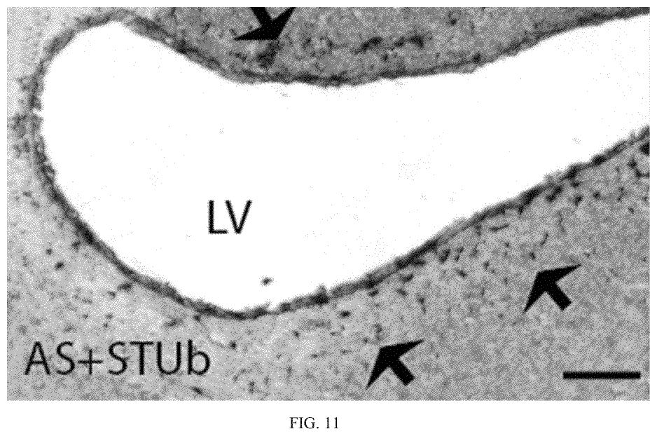

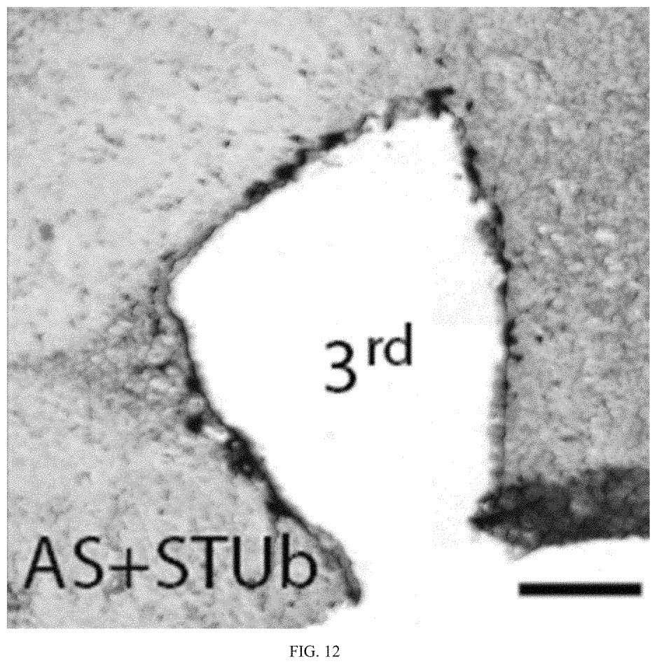

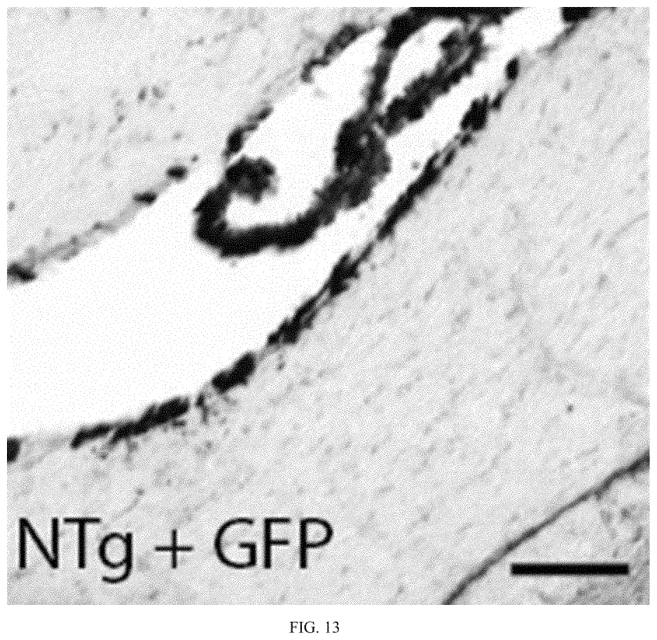

Immunohistochemical analysis of brain slices indicate nontransgenic mice possess relatively high levels of E6-AP, with region-specific staining, seen in FIGS. 5 and 6. In Angelman syndrome-model mice, staining patterns of E6-AP are similar, but the levels of E6-AP are drastically reduced, seen in FIGS. 7 and 8, as expected. Administration of the mouse UBE3A vector to Angelman syndrome model mice did increase levels of E6-AP, though not to the level of nontransgenic mice, as seen in FIGS. 9 and 10. A detailed analysis of the lateral ventricle shows that the injection of UBE3A vector resulted in uptake of the vector by ependymal cells, as seen in FIG. 11. However, in addition to the uptake of UBE3A vector and expression of E6-AP by ependymal cells, adjacent cells in the parenchyma also stained positive for E6-AP, as seen by arrows in the Figure. Moreover, staining was seen in more distal locations, such as the 3d ventricle, seen in FIG. 12. This indicates that E6-AP was being secreted by the transfected cells and successfully uptaken by adjacent cells, confirming that the construct can be used to introduce E6-AP and that the E6-AP construct can be used as a therapeutic to treat global cerebral deficiency in E6-AP expression, such as Angelman syndrome. Control treatment using AAV4-GFP vector did not exhibit uptake of the control protein, as seen in FIG. 13, as only transduction of the ependymal and choroid plexus cells.

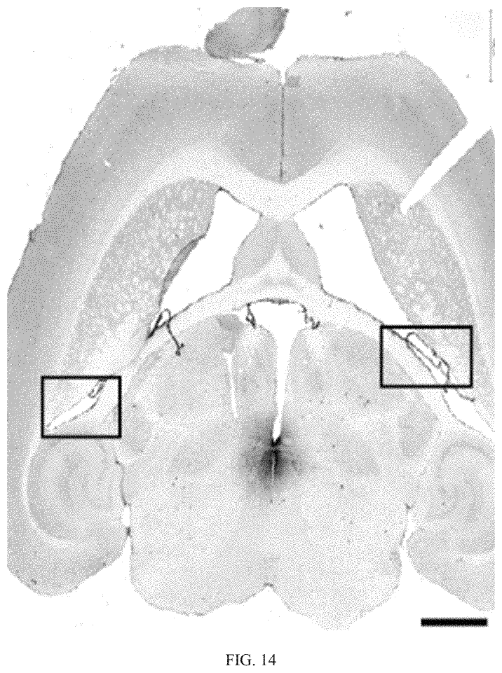

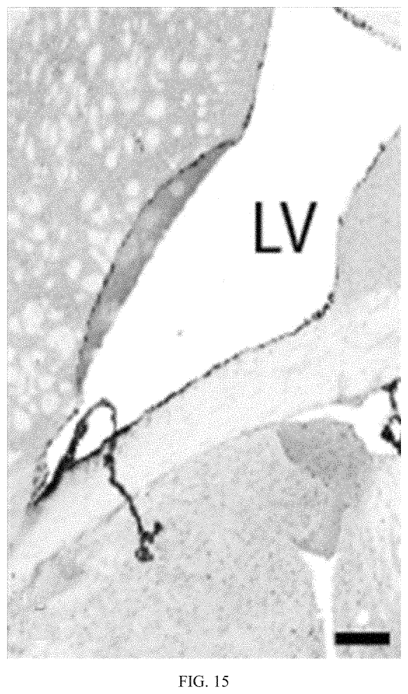

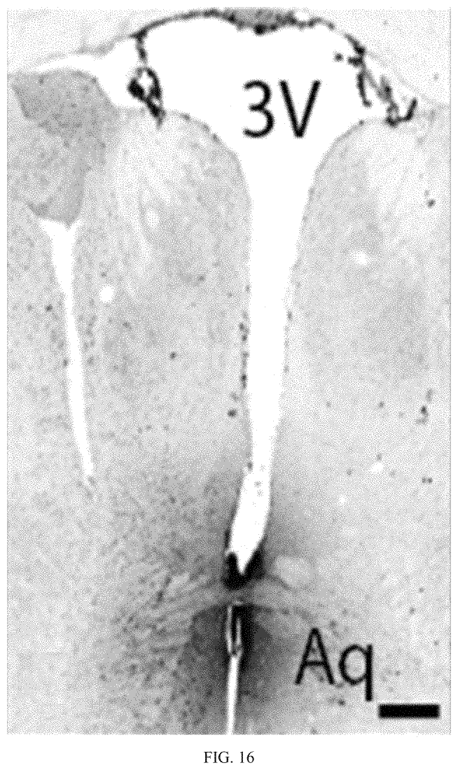

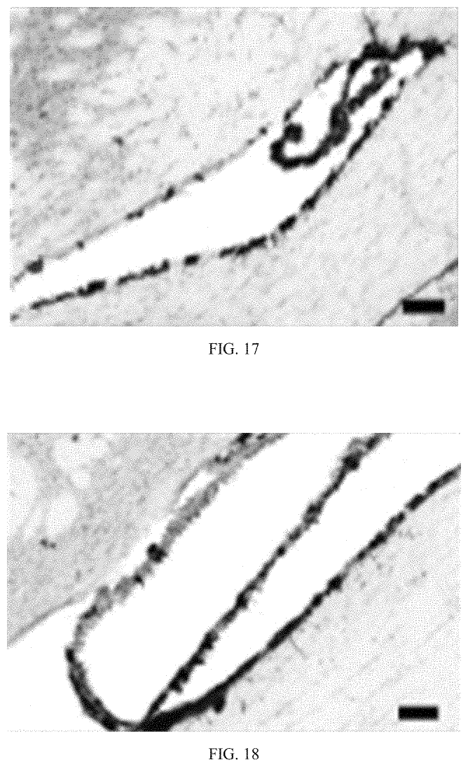

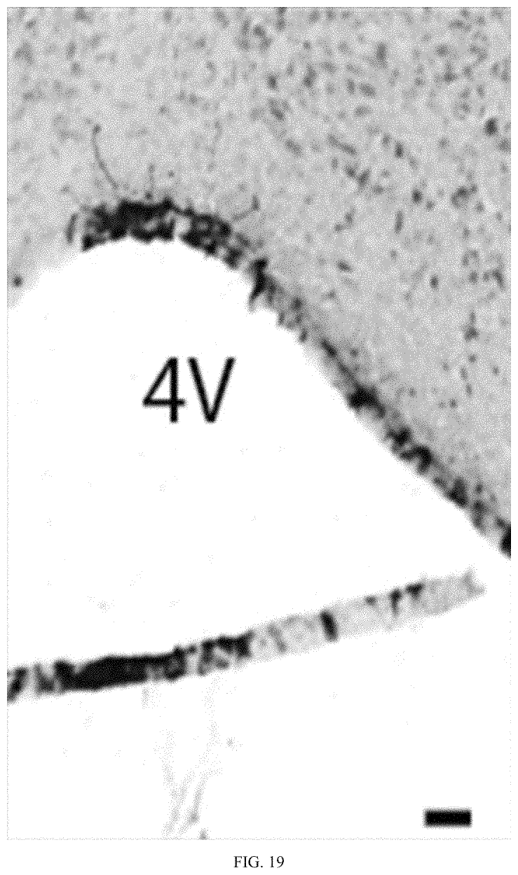

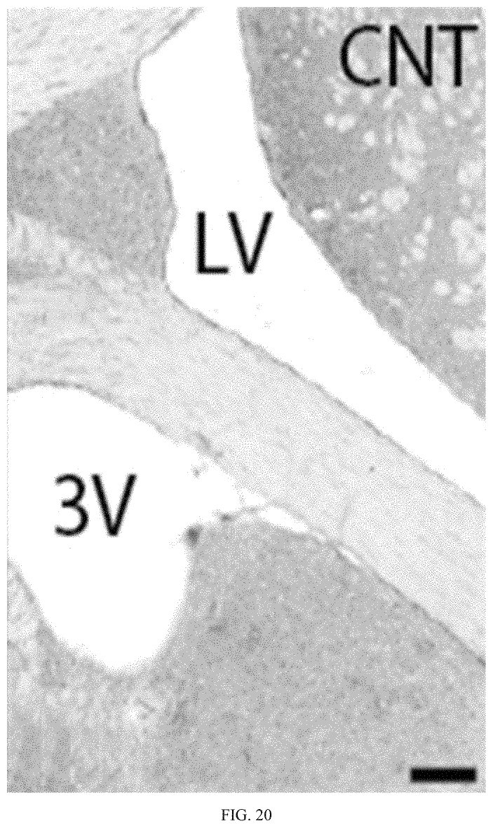

Detailed analysis of the coronal cross sections of Angelman syndrome-model mice confirmed that administration of the UBE3A construct increased levels of E6-AP in and around the lateral ventricle, as seen in FIGS. 14 through 20.

### Example 5—Human UBE3A Vector Construct

A human vector construct was generated using a pTR plasmid. A Homo sapiens UBE3A gene was formed from cDNA (AH005553.1);

(SEQ ID No: 9)
GGAGTAGTTT ACTGAGCCAC TAATCTAAAG TTTAATACTG
TGAGTGAATA CCAGTGAGTA CCTTTGTTAA TGTGGATAAC CAATACTTGG
CTATAGGAAG TTTTTTAGTT GTGTGTTTTA TNACACGTAT TTGACTTTGT
GAATAATTAT GGCTTATAAT GGCTTGTCTG TTGGTATCTA TGTATAGCGT
TTACAGTTTC CTTTAAAAAA CATGCATTGA GTTTTTTAAT AGTCCAACCC
TTAAAATAAA TGTGTTGTAT GGCCACCTGA TCTGACCACT TTCTTTCATG
TTGACATCTT TAATTTTAAA ACTGTTTTAT TTAGTGCTTA AATCTTGTTN
ACAAAATTGT CTTCCTAAGT AATATGTCTA CCTTTTTTTT TGGAATATGG
AATATTTTGC TAACTGTTTC TCAATTGCAT TTTACAGATC AGGAGAACCT
CAGTCTGACG ACATTGAAGC TAGCCGAATG TAAGTGTAAC TTGGTTGAGA
CTGTGGTTCT TATTTTGAGT TGCCCTAGAC TGCTTTAAAT TACGTCACAT
TATTTGGAAA TAATTTCTGG TTAAAAGAAA GGAATCATTT AGCAGTAAAT
GGGAGATAGG AACATACCTA CTTTTTTTCC TATCAGATAA CTCTAAACCT
CGGTAACAGT TTACTAGGTT TCTACTACTA GATAGATAAA TGCACACGCC
TAAATTCTTA GTCTTTTTGC TTCCCTGGTA GCAGTTGTAG GGAAATAGGG
AGGTTGAGGA AAGAGTTTAA CAGTCTCAAC GCCTACCATA TTTAAGGCAT
CAAGTACTAT GTTATAGATA CAGAGATGCG TAATAATTAG TTTTCACCCT
ACAGAAATTT ATATTATACT CAAGAGTGAA AGATGCAGAA GCAAATAATT
TCAGTCACTG AGGTAGAATG GTATCCAAAA TACAATAGTA ACATGAAGGA
GTACTGGAGT ACCAGGTATG CAATAGGAAT CTAGTGTAGA TGGCAGGGAA
GTAAGAGTGG CCAGGAAATG CTAAGTTCAG TCTTGAAATG TGACTGGGAA
TCAGGCAGCT ATCAACTATA AGTCAAATGT TTACAAGCTG TTAAAAATGA
AATACTGATT ATGTAAAAGA AAACCGGATT GATGCTTTAA ATAGACTCAT
TTTCNTAATG CTAATTTTTA AAATGATAGA ATCCTACAAN TCTTAGCTGT
AAACCTTGTG ATTTTTCAGC TGTTGTACTA AACAACTTAA GCACATATAC
CATCAGACAA GCCCCCNTCC CCCCTTTTAA ACCAAAGGAA TGTATACTCT
GTTAATACAG TCAGTAAGCA TTGACATTCT TTATCATAAT ATCCTAGAAA
ATATTTATTA ACTATTTCAC TAGTCAGGAG TTGTGGTAAA TAGTGCATCT
CCATTTTCTA CTTCTCATCT TCATACACAG GTTAATCACT TCAGTGCTTG
ACTAACTTTT GCCTTGATGA TATGTTGAGC TTTGTACTTG AGAGCTGTAC
TAATCACTGT GCTTATTGTT TGAATGTTTG GTACAGGAAG CGAGCAGCTG
CAAAGCATCT AATAGAACGC TACTACCACC AGTTAACTGA GGGCTGTGGA
AATGAAGCCT GCACGAATGA GTTTTGTGCT TCCTGTCCAA CTTTTCTTCG
TATGGATAAT AATGCAGCAG CTATTAAAGC CCTCGAGCTT TATAAGATTA
ATGCAAAACT CTGTGATCCT CATCCCTCCA AGAAAGGAGC AAGCTCAGCT
TACCTTGAGA ACTCGAAAGG TGCCCCCAAC AACTCCTGCT CTGAGATAAA
AATGAACAAG AAAGGCGCTA GAATTGATTT TAAAGGTAAG ATGTTTTATT
TTCAATTGAG AATTGTTGCC TGAAAACCAT GTGGGAGATT TAAATGTATT
AGTTTTTATT TGTTTTTTCT TCTGTGACAT AAAGACATTT TGATATCGTA
GAACCAATTT TTTATTGTGG TAACGGACAG GAATAATAAC TACATTTTAC
AGGTCTAATC ATTGCTAATT AGAAGCAGAT CATATGCCAA AAGTTCATTT
GTTAATAGAT TGATTTGAAC TTTTTAAAAT TCTTAGGAAA AATGTATTAA
GTGGTAGTGA ATCTCCAAAA CTATTTAAGA GCTGTATTAT GATTAATCAG
TACATGACAT ATTGGTTCAT ATTTATAATT AAAGCTATAC ATTAATAGAT
ATCTTGATTA TAAAGAAAGT TTAAACTCAT GATCTTATTA AGAGTTATAC
ATTGTTGAAA GAATGTAAAA GCATGGGTGA GGTCATTGGT ATAGGTAGGT
AGTTCATTGA AAAAAATAGG TAAGCATTAA ATTTTGTTTG CTGAATCTAA
GTATTAGATA CTTTAAGAGT TGTATATCAT AAATGATATT GAGCCTAGAA
TGTTTGGCTG TTTTACTTTT AGAACTTTTT GCAACAGAGT AAACATACAT
ATTATGAAAA TAAATGTTCT CTTTTTTCCT CTGATTTTCT AGATGTGACT
TACTTAACAG AAGAGAAGGT ATATGAAATT CTTGAATTAT GTAGAGAAAG
AGAGGATTAT TCCCCTTTAA TCCGTGTTAT TGGAAGAGTT TTTTCTAGTG
CTGAGGCATT GGTACAGAGC TTCCGGAAAG TTAAACAACA CACCAAGGAA
GAACTGAAAT CTCTTCAAGC AAAAGATGAA GACAAAGATG
AAGATGAAAA GGAAAAAGCT GCATGTTCTG CTGCTGCTAT GGAAGAAGAC
TCAGAAGCAT CTTCCTCAAG GATAGGTGAT AGCTCACAGG GAGACAACAA
TTTGCAAAAA TTAGGCCCTG ATGATGTGTC TGTGGATATT GATGCCATTA
GAAGGGTCTA CACCAGATTG CTCTCTAATG AAAAAATTGA AACTGCCTTT
CTCAATGCAC TTGTATATTT GTCACCTAAC GTGGAATGTG ACTTGACGTA
TCACAATGTA TACTCTCGAG ATCCTAATTA TCTGAATTTG TTCATTATCG
TAATGGAGAA TAGAAATCTC CACAGTCCTG AATATCTGGA AATGGCTTTG
CCATTATTTT GCAAAGCGAT GAGCAAGCTA CCCCTTGCAG CCCAAGGAAA
ACTGATCAGA CTGTGGTCTA AATACAATGC AGACCAGATT CGGAGAATGA
TGGAGACATT TCAGCAACTT ATTACTTATA AAGTCATAAG CAATGAATTT
AACAGTCGAA ATCTAGTGAA TGATGATGAT GCCATTGTTG CTGCTTCGAA
GTGCTTGAAA ATGGTTTACT ATGCAAATGT AGTGGGAGGG GAAGTGGACA
CAAATCACAA TGAAGAAGAT GATGAAGAGC CCATCCCTGA
GTCCAGCGAG CTGACACTTC AGGAACTTTT GGGAGAAGAA
AGAAGAAACA AGAAAGGTCC TCGAGTGGAC CCCCTGGAAA
CTGAACTTGG TGTTAAAACC CTGGATTGTC GAAAACCACT TATCCCTTTT
GAAGAGTTTA TTAATGAACC ACTGAATGAG GTTCTAGAAA TGGATAAAGA
TTATACTTTT TTCAAAGTAG AAACAGAGAA CAAATTCTCT TTTATGACAT
GTCCCTTTAT ATTGAATGCT GTCACAAAGA ATTTGGGATT ATATTATGAC
AATAGAATTC GCATGTACAG TGAACGAAGA ATCACTGTTC TCTACAGCTT
AGTTCAAGGA CAGCAGTTGA ATCCATATTT GAGACTCAAA GTTAGACGTG
ACCATATCAT AGATGATGCA CTTGTCCGGG TAAGTTGGGC TGCTAGATTA
AAAACCTAAT AATGGGGATA TCATGATACA GTTCAGTGAA TTCATTTTAA
AAGTGACTGA AAAAAATGAT ACCATATAGC ATAGGAACAC ATGGACATTT
CTGATCTTAT ATAAGTATTA TACTTTTGTT GTTCCTGTGC AAGTTTATAG
ATGTGTTCTA CAAAGTATCG GTTGTATTAT ATAATGGTCA TGCTATCTTT
GAAAAAGAAT GGGTTTTCTA AATCTTGAAA ACTAAATCCA AAGTTTCTTT
CATTCAGAAG AGAATAGAGT GTTGGACAAA GACCAGAACA
AGAGAAATGT GGAGATACCC AATAATAAGT GTGGATGTGC AGTCTTGAAC
TGGGAGTAAT GGTACAGTAA AACCATACCA TAAAATTATA GGTAGTGTCC
AAAAAATTCC ATCGTGTAAA ATTCAGAGTT GCATTATTGT GGACTTGAAG
AAGCAGTTGT ATGTGGGACG GTATCGATAA GCTTGATATC GAATTCCTGC
AGCCCGGGGG ATCCACTAGT GTGGTAATTA ATACTAAGTC TTACTGTGAG
AGACCATAAA CTGCTTTAGT ATTCAGTGTA TTTTTCTTAA TTGAAATATT
TAACTTATGA CTTAGTAGAT ACTAAGACTT AACCCTTGAG TTTCTATTCT
AATAAAGGAC TACTAATGAA CAATTTTGAG GTTAGACCTC TACTCCATTG
TTTTTGCTGA AATGATTTAG CTGCTTTTCC ATGTCCTGTG TAGTCCAGAC
TTAACACACA AGTAATAAAA TCTTAATTAA TTGTATGTTA ATTTCATAAC
AAATCAGTAA AGTTAGCTTT TTACTATGCT AGTGTCTGTT TTGTGTCTGT
CTTTTTGATT ATCTTTAAGA CTGAATCTTT GTCTTCACTG GCTTTTTATC
AGTTTGCTTT CTGTTTCCAT TTACATACAA AAAGTCAAAA ATTTGTATTT
GTTTCCTAAT CCTACTCCTT GTTTTTATTT TGTTTTTTTC CTGATACTAG
CAATCATCTT CTTTTCATGT TTATCTTTTC AATCACTAGC TAGAGATGAT
CGCTATGGAA AATCCTGCAG ACTTGAAGAA GCAGTTGTAT GTGGAATTTG
AAGGAGAACA AGGAGTTGAT GAGGGAGGTG TTTCCAAAGA ATTTTTTCAG
CTGGTTGTGG AGGAAATCTT CAATCCAGAT ATTGGTAAAT ACATTAGTAA
TGTGATTATG GTGTCGTATC ATCTTTTGAG TTAGTTATTT GTTTATCTTA
CTTTGTAAAT ATTTTCAGCT ATGAAGAGCA GCAAAAGAAG GATTTGGTAT
GGATTACCCA GAATCACACA TCATGACTGA ATTTGTAGGT TTTAGGAACT
GATTTGTATC ACTAATTTAT TCAAATTCTT TTATTTCTTA GAAGGAATAT
TCTAATGAAG GAAATTATCT CTTTGGTAAA CTGAATTGAA AGCACTTTAG
AATGGTATAT TGGAACAGTT GGAGGGATTT CTTTGCTTTT TGTTGTCTAA
AACCATCATC AAACTCACGG TTTTCCTGAC CTGTGAACTT CAAAGAACAA
TGGTTTGAAG AGTATTGAGA GACTGTCTCA CAAGTATGTC ATGCTCAAAG
TTCAGAAACA CTAGCTGATA TCACATTAAT TAGGTTTATT TGCTATAAGA
TTTCTTGGGG CTTAATATAN GTAGTGTTCC CCCAAACTTT TTGAACTCCA
GAACTCTTTT CTGCCCTAAC AGTAGCTACT CAGGAGCTGA GGCAGGAGAA
TTGTTTGAAC CTAGGAGGCA GAGGTTGCAG TGAGCTGAGA TCGTGCCACT
CCAGCCCACC CCTGGGTAAC AGAGCGAGAC TCCATCTCAA AGAAAAAAAT
GAAAAATTGT TTTCAAAAAT AGTACGTGTG GTACAGATAT AAGTAATTAT
ATTTTTATAA ATGAAACACT TTGGAAATGT AGCCATTTTT TGTTTTTTTA
TGTTTATTTT TCAGCTATGG GTGGATAAAG CATGAATATA ACTTTTCTTA
TGTGTTAGTA GAAAATTAGA AAGCTTGAAT TTAATTAACG TATTTTTCTA
CCCGATGCCA CCAAATTACT TACTACTTTA TTCCTTTGGC TTCATAAAAT
TACATATCAC CATTCACCCC AATTTATAGC AGATATATGT GGACATTGTT
TTCTCAAGTG CTAATATAAT AGAAATCAAT GTTGCATGCC TAATTACATA
TATTTTAAAT GTTTTATATG CATAATTATT TTAAGTTTAT ATTTGTATTA
TTCATCAGTC CTTAATAAAA TACAAAAGTA ATGTATTTTT AAAAATCATT
TCTTATAGGT ATGTTCACAT ACGATGAATC TACAAAATTG TTTTGGTTTA
ATCCATCTTC TTTTGAAACT GAGGGTCAGT TTACTCTGAT TGGCATAGTA
CTGGGTCTGG CTATTTACAA TAACTGTATA CTGGATGTAC ATTTTCCCAT
GGTTGTCTAC AGGAAGCTAA TGGGGAAAAA AGGAACTTTT CGTGACTTGG
GAGACTCTCA CCCAGTAAGT TCTTTGTCAT TTTTTTAATT CAGTCTCTTA
GATTTTATTT AAATGCAAAA ATTTAATTTA TGTCAAAATT TTAAAGTTTT
TGTTTAGAAT CTTTGTTGAT ACTCTTATCA ATAAGATAAA AATGTTTTAA
TCTGACCGAA GTACCAGAAA CACTTAAAAA CTCAAAGGGG GACATTTTTA
TATATTGCTG TCAGCACGAA GCTTTCGTAA GATTGATTTC ATAGAGAAGT
GTTTCTAAAC ATTTTGTTTG TGTTTTAGTG AAATCTTAAG AGATAGGTAA
AAATCAGAGT AGCCCTGGCT AAGGGTCTTG GTAGTTACAA CGAGTGTGCC
TGCTCCTACC ACCCCCACCC CCACCTTGAG ACACCACAGA ATTTCTCATA
GAGCACAGTG TGAATTCTAT TGCTAAATTG GTGGTATGGG GTTTCTCAGC
AGAGAATGGG ACATCACAGT GACTGACAAT CTTTCTTTTA TAGGTTGGAA
ACTATTTGGG GGACTGGAGG GATACTGTCT ACACTTTTTA CAATTTTTAT
TGATAAGATT TTTGTTGTCT TCTAAGAAGA GTGATATAAA TTATTTGTTG
TATTTTGTAG TTCTATGGTG GCCTCAATTT ACCATTTCTG GTTGCTAGGT
TCTATATCAG AGTTTAAAAG ATTTATTGGA GTATGAAGGG AATGTGGAAG
ATGACATGAT GATCACTTTC CAGATATCAC AGACAGATCT TTTTGGTAAC
CCAATGATGT ATGATCTAAA GGAAAATGGT GATAAAATTC CAATTACAAA
TGAAAACAGG AAGGTAATAA ATGTTTTTAT GTCACATTTT GTCTCTTCAT
TAACACTTTC AAAGCATGTA TGCTTATAAT TTTTAAAGAA GTATCTAATA
TAGTCTGTAC AAAAAAAAAA CAAGTAACTA AGTTTATGTA AATGCTAGAG
TCCACTTTTC TAAATCTTGG ATATAAGTTG GTATGAAAGC ACACAGTTGG
GCACTAAAGC CCCTTTTAGA GAAAGAGGAC ATGAAGCAGG
AGATAGTTAA TAGCTAAGTG TGGTTGTAGT ATAAAGCAAG AAGCAGGGTG
TTTCTTGTAT TAAGCTGTAA GCAGGAACCT CATGATTAAG GTCTTTATCA
CAGAACAAAT AAAAATTACA TTTAATTTAC ACATGTATAT CCTGTTTGTG
ATAAAAATAC ATTTCTGAAA AGTATACTTT ACGTCAGATT TGGGTTCTAT
TGACTAAAAT GTGTTCATCG GGAATGGGAA TAACCCAGAA CATAACAAGC
AAAAAATTAT GACAAATATA TAGTATACCT TTAAGAAACA TGTTTATATT
GATATAATTT TTTGATTAAA TATTATACAC ACTAAGGGTA CAANGCACAT
TTTCCTTTTA TGANTTNGAT ACAGTAGTTT ATGTGTCAGT CAGATACTTC
CACATTTTTG CTGAACTGGA TACAGTAAGC AGCTTACCAA ATATTCTATG
GTAGAAAACT NGGACTTCCT GGTTTGCTTA AATCAAATAT ATTGTACTCT
CTTAAAACGG TTGGCATTTA TAAATAGATG GATACATGGT TTAAATGTGT
CTGTTNACAT ACCTAGTTGA GAGAACCTAA AGAATTTTCT GCGTCTCCAG
CATTTATATT CAGTTCTGTT TAATACATTA TCGAAATTGA CATTTATAAG
TATGACAGTT TTGTGTATAT GGCCTTTTCA TAGCTTAATA TTGGCTGTAA
CAGAGAATTG TGAAATTGTA AGAAGTAGTT TTCTTTGTAG GTGTAAAATT
GAATTTTTAA GAATATTCTT GACAGTTTTA TGTATATGGC CTTTTCATAG
CTTAATATTG GCTATAACAG AGAATTGTGA AATTGTTAAG AAGTAGGTGT
AAAATTGAAT TTTTAAGAAT ATTCTTGAAT GTTTTTTTCT TGGAAAAATT
AAAAAGCTAT GCAGCCCAAT AACTTGTGTT TTGTTTGCAT AGCATATTAT
AAGAAGTTCT TGTGATTAAT GTTTTCTACA GGAATTTGTC AATCTTTATT
CTGACTACAT TCTCAATAAA TCAGTAGAAA AACAGTTCAA GGCTTTTCGG
AGAGGTTTTC ATATGGTGAC CAATGAATCT CCCTTAAAGT ACTTATTCAG
ACCAGAAGAA ATTGAATTGC TTATATGTGG AAGCCGGGTA
AGAAAGCAGG TGTCTGCAAA AAGTCATGTA TCGATTTATT GTTTGTAATG
ATACAGTAGT ATAGCAGATA ACTAAGACAT ATTTTCTTGA ATTTGCAGAA
TCTAGATTTC CAAGCACTAG AAGAAACTAC AGAATATGAC GGTGGCTATA
CCAGGGACTC TGTTCTGATT AGGTGAGGTA CTTAGTTCTT CAGAGGAAGA
TTTGATTCAC CAAAGGGGTG TGTGATTTTG CTTCAGACCT TTATCTCTAG
GTACTAATTC CCAAATAAGC AAACTCACAA ATTGTCATCT ATATACTTAG
ATTTGTATTT GTAATATAAT CACCATTTTT CAGAGCTAAT CTTGTGATTT
ATTTCATGAA TGAAGTGTTG TTATATATAA GTCTCATGTA ATCTCCTGCA
TTTGGCGTAT GGATTATCTA GTATTCCTCA CTGGTTAGAG TATGCTTACT
GCTGGTTAGA AGATAATTAA AATAAGGCTA CCATGTCTGC AATTTTTCCT
TTCTTTTGAA CTCTGCATTT GTGAACTGTT ACATGGCTTC CCAGGATCAA
GCACTTTTTG AGTGAAATGG TAGTCTTTTA TTTAATTCTT AAGATAATAT
GTCCAGATAC ATACTAGTAT TTCCATTTTA CACCCTAAAA AACTAAGCCC
TGAATTCTCA CAGAAAGATG TAGAGGTTCC CAGTTCTATC TGCTTTTAAA
CAAATGCCCT TACTACTCTA CTGTCTACTT CTGTGTACTA CATCATCGTA
TGTAGTTGTT TGCATTTGGG CCAGTTGGTT GGGGCAGGGG TCTTTTTTTC
TTTTGTCCCT TAATCTGTAT CACTTTTTCC TCCCAAAGTT GAGTTAAAGG
ATGAGTAGAC CAGGAGAATA AAGGAGAAAG GATAAATAAA
ATATATACCC AAAGGCACCT GGAGTTAATT TTTCCAAATA TTCATTTCAG
TCTTTTTCAA TTCATAGGAT TTTGTCTTTT GCTCATTACT GACTGCATAA
TGTGATTATA CCATAGTTTA AATAGTCACT TCCTGTTACT ACACACTTGG
GTTTTCTCAA TTTTTTACTA TTGTAGTACT AATATTTTAC TATATTGTAA
TCTAATCCAA ATTTTTACGT ATTCAGAGCT GTTCAGGATA AATTTGCTTG
GAAATTTTTA AATCACCAGA AGTGATACTA TCCTGATAAT TAACTTCCAA
GTTGTCTCTT AATATAGTTT TAATGCAAAT CATAAGCTTA TGTTAGTACC
AGTCATAATG AATGCCAAAC TGAAACCAGT ATTGTATTTT TTCTCATTAG
GGAGTTCTGG GAAATCGTTC ATTCATTTAC AGATGAACAG AAAAGACTCT
TCTTGCAGTT TACAACGGGC ACAGACAGAG CACCTGTGGG AGGACTAGGA
AAATTAAAGA TGATTATAGC CAAAAATGGC CCAGACACAG
AAAGGTAGGT AATTATTAAC TTGTGACTGT ATACCTACCG AAAACCTTGC
ATTCCTCGTC ACATACATAT GAACTGTCTT TATAGTTTCT GAGCACATTC
GTGATTTTAT ATACAAATCC CCAAATCATA TTAGACAATT GAGAAAATAC
TTTGCTGTCA TTGTGTGAGG AAACTTTTAA GAAATTGCCC TAGTTAAAAA
TTATTATGGG GCTCACATTG GTTTGGAATC AAATTAGTGT GATTCATTTA
CTTTTTTGAT TCCCAGCTTG TTAATTGAAA GCCATATAAC ATGATCATCT
ATTTAGAATG GTTACATTGA GGCTCGGAAG ATTATCATTT GATTGTGCTA
GAATCCTGTT ATCAAATCAT TTTCTTAGTC ATATTGCCAG CAGTGTTTCT
AATAAGCATT TAAGAGCACA CACTTTGCAG TCTTGTAAAA CAGGTTTGAG
TATTTTCTCC ACCTTAGAGG AAGTTACTTG ACTTCTCAGT GACCTAACCT
CTAAAGTGCA TTTACTGATG TCCTCTCTGT GGTTTTGTTG TGGAAAGATT
TAGTTAAATG AACTGTAAGA ATTCAGTACC TAAAATGGTA TCTGTTATGT
AGTAAAAACT CAATGGATAC AGTATCTTAT CATCGTCACT AGCTTTGAGT
AATTTATAGG ATAAAGGCAA CTTGGTAGTT ACACAACAAA AAGTTTATGA
TTTGCATTAA TGTATAGTTT GCATTGCAGA CCGTCTCAAC TATATACAAT
CTAAAAATAG GAGCATTTAA TTCTAAGTGT ATTTCCCATG ACTTACAGTT
TTCCTGTTTT TTTCCCCTTT TCTCTATTTA GGTTACCTAC ATCTCATACT
TGCTTTAATG TGCTTTTACT TCCGGAATAC TCAAGCAAAG AAAAACTTAA
AGAGAGATTG TTGAAGGCCA TCACGTATGC CAAAGGATTT GGCATGCTGT
AAAACAAAAC AAAACAAAAT AAAACAAAAA AAAGGAAGGA
AAAAAAAAGA AAAAATTTAA AAAATTTTAA AAATATAACG
AGGGATAAAT TTT (AH005553.1), which encodes for;
(SEQ ID No: 10)
MKRAAAKHLIERYYHQLTEGCGNEACTNEFCASCPTFLRMDNNAAAIKA
LELYKINAKLCDPHPSKKGASSAYLENSKGAPNNSCSEIKMNKKGARIDFKDVT
YLTEEKVYEILELCREREDYSPLIRVIGRVFSSAEALVQSFRKVKQHTKEELKSL
QAKDEDKDEDEKEKAACSAAAMEEDSEASSSRIGDSSQGDNNLQKLGPDDVS
VDIDAIRRVYTRLLSNEKIETAFLNALVYLSPNVECDLTYHNVYSRDPNYLNLFI
IVMENRNLHSPEYLEMALPLFCKAMSKLPLAAQGKLIRLWSKYNADQIRRMME
TFQQLITYKVISNEFNSRNLVNDDDAIVAASKCLKMVYYANVVGGEVDTNHNE
EDDEEPIPESSELTLQELLGEERRNKKGPRVDPLETELGVKTLDCRKPLIPFEEFI
NEPLNEVLEMDKDYTFFKVETENKFSFMTCPFILNAVTKNLGLYYDNRIRMYSE
RRITVLYSLVQGQQLNPYLRLKVRRDHIIDDALVRLEMIAMENPADLKKQLYV
EFEGEQGVDEGGVSKEFFQLVVEEIFNPDIGMFTYDESTKLFWFNPSSFETEGQF
TLIGIVLGLAIYNNCILDVHFPMVVYRKLMGKKGTFRDLGDSHPVLYQSLKDLL
EYEGNVEDDMMITFQISQTDLFGNPMMYDLKENGDKIPITNENRKEFVNLYSD
YILNKSVEKQFKAFRRGFHMVTNESPLKYLFRPEEIELLICGSRNLDFQALEETT
EYDGGYTRDSVLIREFWEIVHSFTDEQKRLFLQFTTGTDRAPVGGLGKLKMIIA
KNGPDTERLPTSHTCFNVLLLPEYSSKEKLKERLLKAITYAKGFGML (NP 570853.1).

The cDNA was subcloned and sequenced. The UBE3A, version 1 gene (hUBEv1) (SEQ ID No: 9) was fused to one of three genes encoding a secretion signaling peptide, based on GDNF;

(SEQ ID No: 2)
ATGAAGTTATGGGATGTCGTGGCTGTCTGCCTGGTGCTGCTCCACACC
GCGTCCGCC,

from insulin protein;

(SEQ ID No: 11)
ATGGCCCTGTGGATGCGCCTCCTGCCCCTGCTGGCGCTGCTGGCCCTCT
GGGGACCTGACCCAGCCGCAGCC
(AH002844.2),

or from IgK;

(SEQ ID No: 12)
ATGGAGACAGACACACTCCTGCTATGGGTACTGCTGCTCTGGGTTCCA
GGTTCCACTGGT
(NG 000834.1).

The construct was inserted into the hSTUb vector, under a CMV chicken-beta actin hybrid promoter or human ubiquitin c promoter. Woodchuck hepatitis post-transcriptional regulatory element (WPRE) is present to increase expression levels.

The UBE3A-seretion signal construct was then attached to a cellular uptake peptide (cell penetrating peptide); either a

| HIV TAT sequence | |
|---|---|
| YGRKKRRQRRR; | (SEQ ID No. 8) |
| or | |
| HIV TATk sequence | |
| YARKAARQARA. | (SEQ ID No. 13) |

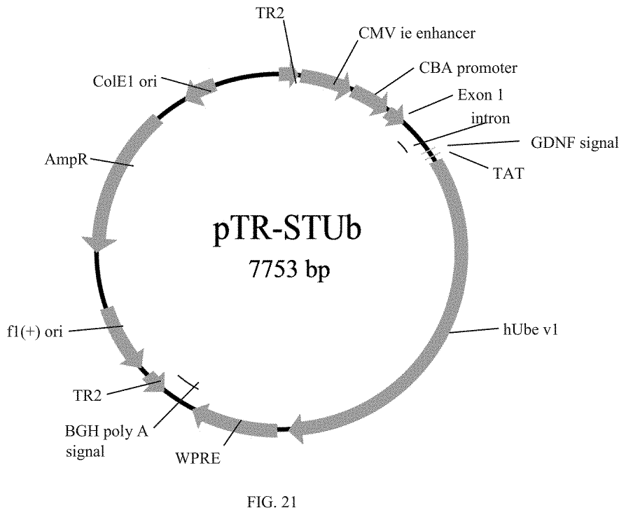

The human UBE3A vector, seen in FIG. 21, is then transformed into E. coli using the heat shock method described in Example 2. The transformed E. coli were expanded in broth containing ampicillin to select for the vector and collect large amounts of vector.

Other sequences of UBE3A include variants 1, 2, or 3, seen below;

H sapiens UBE3A variant 1:

(SEQ ID No: 14)
ACAGTATGAC ATCTGATGCT GGAGGGTCGC ACTTTCACAA
ATGAGTCAGC TGGTACATGG GGTTATCATC AATTTTTAGC
TCTTCTGTCT GGGAGATACA AGTTTGGAAG CAATCTTGGG
GTACTTACCC ACAAGGCTGG TGGAGACCAG ATCAGGAGAA
CCTCAGTCTG ACGACATTGA AGCTAGCCGA ATGAAGCGAG
CAGCTGCAAA GCATCTAATA GAACGCTACT ACCACCAGTT
AACTGAGGGC TGTGGAAATG AAGCCTGCAC GAATGAGTTT
TGTGCTTCCT GTCCAACTTT TCTTCGTATG GATAATAATG
CAGCAGCTAT TAAAGCCCTC GAGCTTTATA AGATTAATGC
AAAACTCTGT GATCCTCATC CCTCCAAGAA AGGAGCAAGC
TCAGCTTACC TTGAGAACTC GAAAGGTGCC CCCAACAACT
CCTGCTCTGA GATAAAAATG AACAAGAAAG GCGCTAGAAT
TGATTTTAAA GATGTGACTT ACTTAACAGA AGAGAAGGTA
TATGAAATTC TTGAATTATG TAGAGAAAGA GAGGATTATT
CCCCTTTAAT CCGTGTTATT GGAAGAGTTT TTTCTAGTGC
TGAGGCATTG GTACAGAGCT TCCGGAAAGT TAAACAACAC
ACCAAGGAAG AACTGAAATC TCTTCAAGCA AAAGATGAAG
ACAAAGATGA GGATGAAAAG GAAAAAGCTG CATGTTCTGC
TGCTGCTATG GAAGAAGACT CAGAAGCATC TTCCTCAAGG
ATAGGTGATA GCTCACAGGG AGACAACAAT TTGCAAAAAT
TAGGCCCTGA TGATGTGTCT GTGGATATTG ATGCCATTAG
AAGGGTCTAC ACCAGATTGC TCTCTAATGA AAAAATTGAA
ACTGCCTTTC TCAATGCACT TGTATATTTG TCACCTAACG
TGGAATGTGA CTTGACGTAT CACAATGTAT ACTCTCGAGA
TCCTAATTAT CTGAATTTGT TCATTATCGT AATGGAGAAT
AGAAATCTCC ACAGTCCTGA ATATCTGGAA ATGGCTTTGC
CATTATTTTG CAAAGCGATG AGCAAGCTAC CCCTTGCAGC
CCAAGGAAAA CTGATCAGAC TGTGGTCTAA ATACAATGCA
GACCAGATTC GGAGAATGAT GGAGACATTT CAGCAACTTA
TTACTTATAA AGTCATAAGC AATGAATTTA ACAGTCGAAA
TCTAGTGAAT GATGATGATG CCATTGTTGC TGCTTCGAAG
TGCTTGAAAA TGGTTTACTA TGCAAATGTA GTGGGAGGGG
AAGTGGACAC AAATCACAAT GAAGAAGATG ATGAAGAGCC
CATCCCTGAG TCCAGCGAGC TGACACTTCA GGAACTTTTG
GGAGAAGAAA GAAGAAACAA GAAAGGTCCT CGAGTGGACC
CCCTGGAAAC TGAACTTGGT GTTAAAACCC TGGATTGTCG
AAAACCACTT ATCCCTTTTG AAGAGTTTAT TAATGAACCA
CTGAATGAGG TTCTAGAAAT GGATAAAGAT TATACTTTTT
TCAAAGTAGA AACAGAGAAC AAATTCTCTT TTATGACATG
TCCCTTTATA TTGAATGCTG TCACAAAGAA TTTGGGATTA
TATTATGACA ATAGAATTCG CATGTACAGT GAACGAAGAA
TCACTGTTCT CTACAGCTTA GTTCAAGGAC AGCAGTTGAA
TCCATATTTG AGACTCAAAG TTAGACGTGA CCATATCATA
GATGATGCAC TTGTCCGGCT AGAGATGATC GCTATGGAAA
ATCCTGCAGA CTTGAAGAAG CAGTTGTATG TGGAATTTGA
AGGAGAACAA GGAGTTGATG AGGGAGGTGT TTCCAAAGAA
TTTTTTCAGC TGGTTGTGGA GGAAATCTTC AATCCAGATA
TTGGTATGTT CACATACGAT GAATCTACAA AATTGTTTTG
GTTTAATCCA TCTTCTTTTG AAACTGAGGG TCAGTTTACT
CTGATTGGCA TAGTACTGGG TCTGGCTATT TACAATAACT
GTATACTGGA TGTACATTTT CCCATGGTTG TCTACAGGAA
GCTAATGGGG AAAAAAGGAA CTTTTCGTGA CTTGGGAGAC
TCTCACCCAG TTCTATATCA GAGTTTAAAA GATTTATTGG
AGTATGAAGG GAATGTGGAA GATGACATGA TGATCACTTT
CCAGATATCA CAGACAGATC TTTTTGGTAA CCCAATGATG
TATGATCTAA AGGAAAATGG TGATAAAATT CCAATTACAA
ATGAAAACAG GAAGGAATTT GTCAATCTTT ATTCTGACTA
CATTCTCAAT AAATCAGTAG AAAAACAGTT CAAGGCTTTT
CGGAGAGGTT TTCATATGGT GACCAATGAA TCTCCCTTAA
AGTACTTATT CAGACCAGAA GAAATTGAAT TGCTTATATG
TGGAAGCCGG AATCTAGATT TCCAAGCACT AGAAGAAACT
ACAGAATATG ACGGTGGCTA TACCAGGGAC TCTGTTCTGA
TTAGGGAGTT CTGGGAAATC GTTCATTCAT TTACAGATGA
ACAGAAAAGA CTCTTCTTGC AGTTTACAAC GGGCACAGAC
AGAGCACCTG TGGGAGGACT AGGAAAATTA AAGATGATTA
TAGCCAAAAA TGGCCCAGAC ACAGAAAGGT TACCTACATC
TCATACTTGC TTTAATGTGC TTTTACTTCC GGAATACTCA
AGCAAAGAAA AACTTAAAGA GAGATTGTTG AAGGCCATCA
CGTATGCCAA AGGATTTGGC ATGCTGTAAA ACAAAACAAA
ACAAAAT
(AK291405.1);

H sapiens UBE3A variant 2;

(SEQ ID No: 15)
AGCCAGTCCT CCCGTCTTGC GCCGCGGCCG CGAGATCCGT
GTGTCTCCCA AGATGGTGGC GCTGGGCTCG GGGTGACTAC
AGGAGACGAC GGGGCCTTTT CCCTTCGCCA GGACCCGACA
CACCAGGCTT CGCTCGCTCG CGCACCCCTC CGCCGCGTAG
CCATCCGCCA GCGCGGGCGC CCGCCATCCG CCGCCTACTT
ACGCTTCACC TCTGCCGACC CGGCGCGCTC GGCTGCGGGC
GGCGGCGCCT CCTTCGGCTC CTCCTCGGAA TAGCTCGCGG
CCTGTAGCCC CTGGCAGGAG GGCCCCTCAG CCCCCCGGTG
TGGACAGGCA GCGGCGGCTG GCGACGAACG CCGGGATTTC
GGCGGCCCCG GCGCTCCCTT TCCCGGCCTC GTTTTCCGGA
TAAGGAAGCG CGGGTCCCGC ATGAGCCCCG GCGGTGGCGG
CAGCGAAAGA GAACGAGGCG GTGGCGGGCG GAGGCGGCGG
GCGAGGGCGA CTACGACCAG TGAGGCGGCC GCCGCAGCCC
AGGCGCGGGG GCGACGACAG GTTAAAAATC TGTAAGAGCC
TGATTTTAGA ATTCACCAGC TCCTCAGAAG TTTGGCGAAA
TATGAGTTAT TAAGCCTACG CTCAGATCAA GGTAGCAGCT
AGACTGGTGT GACAACCTGT TTTTAATCAG TGACTCAAAG
CTGTGATCAC CCTGATGTCA CCGAATGGCC ACAGCTTGTA
AAAGAGAGTT ACAGTGGAGG TAAAAGGAGT GGCTTGCAGG
ATGGAGAAGC TGCACCAGTG TTATTGGAAA TCAGGAGAAC
CTCAGTCTGA CGACATTGAA GCTAGCCGAA TGAAGCGAGC
AGCTGCAAAG CATCTAATAG AACGCTACTA CCACCAGTTA
ACTGAGGGCT GTGGAAATGA AGCCTGCACG AATGAGTTTT
GTGCTTCCTG TCCAACTTTT CTTCGTATGG ATAATAATGC
AGCAGCTATT AAAGCCCTCG AGCTTTATAA GATTAATGCA
AAACTCTGTG ATCCTCATCC CTCCAAGAAA GGAGCAAGCT
CAGCTTACCT TGAGAACTCG AAAGGTGCCC CCAACAACTC
CTGCTCTGAG ATAAAAATGA ACAAGAAAGG CGCTAGAATT
GATTTTAAAG ATGTGACTTA CTTAACAGAA GAGAAGGTAT
ATGAAATTCT TGAATTATGT AGAGAAAGAG AGGATTATTC
CCCTTTAATC CGTGTTATTG GAAGAGTTTT TTCTAGTGCT
GAGGCATTGG TACAGAGCTT CCGGAAAGTT AAACAACACA
CCAAGGAAGA ACTGAAATCT CTTCAAGCAA AAGATGAAGA
CAAAGATGAA GATGAAAAGG AAAAAGCTGC ATGTTCTGCT
GCTGCTATGG AAGAAGACTC AGAAGCATCT TCCTCAAGGA
TAGGTGATAG CTCACAGGGA GACAACAATT TGCAAAAATT
AGGCCCTGAT GATGTGTCTG TGGATATTGA TGCCATTAGA
AGGGTCTACA CCAGATTGCT CTCTAATGAA AAAATTGAAA
CTGCCTTTCT CAATGCACTT GTATATTTGT CACCTAACGT
GGAATGTGAC TTGACGTATC ACAATGTATA CTCTCGAGAT
CCTAATTATC TGAATTTGTT CATTATCGTA ATGGAGAATA
GAAATCTCCA CAGTCCTGAA TATCTGGAAA TGGCTTTGCC
ATTATTTTGC AAAGCGATGA GCAAGCTACC CCTTGCAGCC
CAAGGAAAAC TGATCAGACT GTGGTCTAAA TACAATGCAG
ACCAGATTCG GAGAATGATG GAGACATTTC AGCAACTTAT
TACTTATAAA GTCATAAGCA ATGAATTTAA CAGTCGAAAT
CTAGTGAATG ATGATGATGC CATTGTTGCT GCTTCGAAGT
GCTTGAAAAT GGTTTACTAT GCAAATGTAG TGGGAGGGGA
AGTGGACACA AATCACAATG AAGAAGATGA TGAAGAGCCC
ATCCCTGAGT CCAGCGAGCT GACACTTCAG GAACTTTTGG
GAGAAGAAAG AAGAAACAAG AAAGGTCCTC GAGTGGACCC
CCTGGAAACT GAACTTGGTG TTAAAACCCT GGATTGTCGA
AAACCACTTA TCCCTTTTGA AGAGTTTATT AATGAACCAC
TGAATGAGGT TCTAGAAATG GATAAAGATT ATACTTTTTT
CAAAGTAGAA ACAGAGAACA AATTCTCTTT TATGACATGT
CCCTTTATAT TGAATGCTGT CACAAAGAAT TTGGGATTAT
ATTATGACAA TAGAATTCGC ATGTACAGTG AACGAAGAAT
CACTGTTCTC TACAGCTTAG TTCAAGGACA GCAGTTGAAT
CCATATTTGA GACTCAAAGT TAGACGTGAC CATATCATAG
ATGATGCACT TGTCCGGCTA GAGATGATCG CTATGGAAAA
TCCTGCAGAC TTGAAGAAGC AGTTGTATGT GGAATTTGAA
GGAGAACAAG GAGTTGATGA GGGAGGTGTT TCCAAAGAAT
TTTTTCAGCT GGTTGTGGAG GAAATCTTCA ATCCAGATAT
TGGTATGTTC ACATACGATG AATCTACAAA ATTGTTTTGG
TTTAATCCAT CTTCTTTTGA AACTGAGGGT CAGTTTACTC
TGATTGGCAT AGTACTGGGT CTGGCTATTT ACAATAACTG
TATACTGGAT GTACATTTTC CCATGGTTGT CTACAGGAAG
CTAATGGGGA AAAAAGGAAC TTTTCGTGAC TTGGGAGACT
CTCACCCAGT TCTATATCAG AGTTTAAAAG ATTTATTGGA
GTATGAAGGG AATGTGGAAG ATGACATGAT GATCACTTTC
CAGATATCAC AGACAGATCT TTTTGGTAAC CCAATGATGT
ATGATCTAAA GGAAAATGGT GATAAAATTC CAATTACAAA
TGAAAACAGG AAGGAATTTG TCAATCTTTA TTCTGACTAC
ATTCTCAATA AATCAGTAGA AAAACAGTTC AAGGCTTTTC
GGAGAGGTTT TCATATGGTG ACCAATGAAT CTCCCTTAAA
GTACTTATTC AGACCAGAAG AAATTGAATT GCTTATATGT
GGAAGCCGGA ATCTAGATTT CCAAGCACTA GAAGAAACTA
CAGAATATGA CGGTGGCTAT ACCAGGGACT CTGTTCTGAT
TAGGGAGTTC TGGGAAATCG TTCATTCATT TACAGATGAA
CAGAAAAGAC TCTTCTTGCA GTTTACAACG GGCACAGACA
GAGCACCTGT GGGAGGACTA GGAAAATTAA AGATGATTAT
AGCCAAAAAT GGCCCAGACA CAGAAAGGTT ACCTACATCT
CATACTTGCT TTAATGTGCT TTTACTTCCG GAATACTCAA
GCAAAGAAAA ACTTAAAGAG AGATTGTTGA AGGCCATCAC
GTATGCCAAA GGATTTGGCA TGCTGTAAAA CAAAACAAAA
CAAAATAAAA CAAAAAAAAG GAAGGAAAAA AAAAGAAAAA
ATTTAAAAAA TTTTAAAAAT ATAACGAGGG ATAAATTTTT
GGTGGTGATA GTGTCCCAGT ACAAAAAGGC TGTAAGATAG
TCAACCACAG TAGTCACCTA TGTCTGTGCC TCCCTTCTTT
ATTGGGGACA TGTGGGCTGG AACAGCAGAT TTCAGCTACA
TATATGAACA AATCCTTTAT TATTATTATA ATTATTTTTT
TGCGTGAAAG TGTTACATAT TCTTTCACTT GTATGTACAG
AGAGGTTTTT CTGAATATTT ATTTTAAGGG TTAAATCACT
TTTGCTTGTG TTTATTACTG CTTGAGGTTG AGCCTTTTGA
GTATTTAAAA AATATATACC AACAGAACTA CTCTCCCAAG
GAAAATATTG CCACCATTTG TAGACCACGT AACCTTCAAG
TATGTGCTAC TTTTTTGTCC CTGTATCTAA CTCAAATCAG
GAACTGTATT TTTTTTAATG ATTTGCTTTT GAAACTTGAA
GTCTTGAAAA CAGTGTGATG CAATTACTGC TGTTCTAGCC
CCCAAAGAGT TTTCTGTGCA AAATCTTGAG AATCAATCAA
TAAAGAAAGA TGGAAGGAAG GGAGAAATTG GAATGTTTTA
ACTGCAGCCC TCAGAACTTT AGTAACAGCA CAACAAATTA
AAAACAAAAA CAACTCATGC CACAGTATGT CGTCTTCATG
TGTCTTGCAA TGAACTGTTT CAGTAGCCAA TCCTCTTTCT
TAGTATATGA AAGGACAGGG ATTTTTGTTC TTGTTGTTCT
CGTTGTTGTT TTAAGTTTAC TGGGGAAAGT GCATTTGGCC
AAATGAAATG GTAGTCAAGC CTATTGCAAC AAAGTTAGGA
AGTTTGTTGT TTGTTTATTA TAAACAAAAA GCATGTGAAA
GTGCACTTAA GATAGAGTTT TTATTAATTA CTTACTTATT
ACCTAGATTT TAAATAGACA ATCCAAAGTC TCCCCTTCGT
GTTGCCATCA TCTTGTTGAA TCAGCCATTT TATCGAGGCA
CGTGATCAGT GTTGCAACAT AATGAAAAAG ATGGCTACTG
TGCCTTGTGT TACTTAATCA TACAGTAAGC TGACCTGGAA
ATGAATGAAA CTATTACTCC TAAGAATTAC ATTGTATAGC
CCCACAGATT AAATTTAATT AATTAATTCA AAACATGTTA
AACGTTACTT TCATGTACTA TGGAAAAGTA CAAGTAGGTT
TACATTACTG ATTTCCAGAA GTAAGTAGTT TCCCCTTTCC
TAGTCTTCTG TGTATGTGAT GTTGTTAATT TCTTTTATTG
CATTATAAAA TAAAAGGATT ATGTATTTTT AACTAAGGTG
AGACATTGAT ATATCCTTTT GCTACAAGCT ATAGCTAATG
TGCTGAGCTT GTGCCTTGGT GATTGATTGA TTGATTGACT
GATTGTTTTA ACTGATTACT GTAGATCAAC CTGATGATTT
GTTTGTTTGA AATTGGCAGG AAAAATGCAG CTTTCAAATC
ATTGGGGGGA GAAAAAGGAT GTCTTTCAGG ATTATTTTAA
TTAATTTTTT TCATAATTGA GACAGAACTG TTTGTTATGT
ACCATAATGC TAAATAAAAC TGTGGCACTT TTCACCATAA
TTTAATTTAG TGGAAAAAGA AGACAATGCT TTCCATATTG
TGATAAGGTA ACATGGGGTT TTTCTGGGCC AGCCTTTAGA
ACACTGTTAG GGTACATACG CTACCTTGAT GAAAGGGACC
TTCGTGCAAC TGTAGTCATC TTAAAGGCTT CTCATCCACT
GTGCTTCTTA ATGTGTAATT AAAGTGAGGA GAAATTAAAT
ACTCTGAGGG CGTTTTATAT AATAAATTCG TGAAGA
(NM 000462.4), which encodes the protein:
(SEQ ID No: 16)
MEKLHQCYWK SGEPQSDDIE ASRMKRAAAK HLIERYYHQL
TEGCGNEACT NEFCASCPTF LRMDNNAAAI KALELYKINA
KLCDPHPSKK GASSAYLENS KGAPNNSCSE IKMNKKGARI
DFKDVTYLTE EKVYEILELC REREDYSPLI RVIGRVFSSA
EALVQSFRKV KQHTKEELKS LQAKDEDKDE DEKEKAACSA
AAMEEDSEAS SSRIGDSSQG DNNLQKLGPD DVSVDIDAIR
RVYTRLLSNE KIETAFLNAL VYLSPNVECD LTYHNVYSRD
PNYLNLFIIV MENRNLHSPE YLEMALPLFC KAMSKLPLAA
QGKLIRLWSK YNADQIRRMM ETFQQLITYK VISNEFNSRN
LVNDDDAIVA ASKCLKMVYY ANVVGGEVDT NHNEEDDEEP
IPESSELTLQ ELLGEERRNK KGPRVDPLET ELGVKTLDCR
KPLIPFEEFI NEPLNEVLEM DKDYTFFKVE TENKFSFMTC
PFILNAVTKN LGLYYDNRIR MYSERRITVL YSLVQGQQLN
PYLRLKVRRD HIIDDALVRL EMIAMENPAD LKKQLYVEFE
GEQGVDEGGV SKEFFQLVVE EIFNPDIGMF TYDESTKLFW
FNPSSFETEG QFTLIGIVLG LAIYNNCILD VHFPMVVYRK
LMGKKGTFRD LGDSHPVLYQ SLKDLLEYEG NVEDDMMITF
QISQTDLFGN PMMYDLKENG DKIPITNENR KEFVNLYSDY
ILNKSVEKQF KAFRRGFHMV TNESPLKYLF RPEEIELLIC
GSRNLDFQAL EETTEYDGGY TRDSVLIREF WEIVHSFTDE
QKRLFLQFTT GTDRAPVGGL GKLKMIIAKN GPDTERLPTS
HTCFNVLLLP EYSSKEKLKE RLLKAITYAK GFGML
(NP 000453.2);

H sapiens UBE3A variant 3

(SEQ ID No: 17)
TTTTTCCGGA TAAGGAAGCG CGGGTCCCGC ATGAGCCCCG
GCGGTGGCGG CAGCGAAAGA GAACGAGGCG GTGGCGGGCG
GAGGCGGCGG GCGAGGGCGA CTACGACCAG TGAGGCGGCC
GCCGCAGCCC AGGCGCGGGG GCGACGACAG GTTAAAAATC
TGTAAGAGCC TGATTTTAGA ATTCACCAGC TCCTCAGAAG
TTTGGCGAAA TATGAGTTAT TAAGCCTACG CTCAGATCAA
GGTAGCAGCT AGACTGGTGT GACAACCTGT TTTTAATCAG
TGACTCAAAG CTGTGATCAC CCTGATGTCA CCGAATGGCC
ACAGCTTGTA AAAGATCAGG AGAACCTCAG TCTGACGACA
TTGAAGCTAG CCGAATGAAG CGAGCAGCTG CAAAGCATCT
AATAGAACGC TACTACCACC AGTTAACTGA GGGCTGTGGA
AATGAAGCCT GCACGAATGA GTTTTGTGCT TCCTGTCCAA
CTTTTCTTCG TATGGATAAT AATGCAGCAG CTATTAAAGC
CCTCGAGCTT TATAAGATTA ATGCAAAACT CTGTGATCCT
CATCCCTCCA AGAAAGGAGC AAGCTCAGCT TACCTTGAGA
ACTCGAAAGG TGCCCCCAAC AACTCCTGCT CTGAGATAAA
AATGAACAAG AAAGGCGCTA GAATTGATTT TAAAGATGTG
ACTTACTTAA CAGAAGAGAA GGTATATGAA ATTCTTGAAT
TATGTAGAGA AAGAGAGGAT TATTCCCCTT TAATCCGTGT
TATTGGAAGA GTTTTTTCTA GTGCTGAGGC ATTGGTACAG
AGCTTCCGGA AAGTTAAACA ACACACCAAG GAAGAACTGA
AATCTCTTCA AGCAAAAGAT GAAGACAAAG ATGAAGATGA
AAAGGAAAAA GCTGCATGTT CTGCTGCTGC TATGGAAGAA
GACTCAGAGG CATCTTCCTC AAGGATAGGT GATAGCTCAC
AGGGAGACAA CAATTTGCAA AAATTAGGCC CTGATGATGT
GTCTGTGGAT ATTGATGCCA TTAGAAGGGT CTACACCAGA
TTGCTCTCTA ATGAAAAAAT TGAAACTGCC TTTCTCAATG
CACTTGTATA TTTGTCACCT AACGTGGAAT GTGACTTGAC
GTATCACAAT GTATACTCTC GAGATCCTAA TTATCTGAAT
TTGTTCATTA TCGTAATGGA GAATAGAAAT CTCCACAGTC
CTGAATATCT GGAAATGGCT TTGCCATTAT TTTGCAAAGC
GATGAGCAAG CTACCCCTTG CAGCCCAAGG AAAACTGATC
AGACTGTGGT CTAAATACAA TGCAGACCAG ATTCGGAGAA
TGATGGAGAC ATTTCAGCAA CTTATTACTT ATAAAGTCAT
AAGCAATGAA TTTAACAGTC GAAATCTAGT GAATGATGAT
GATGCCATTG TTGCTGCTTC GAAGTGCTTG AAAATGGTTT
ACTATGCAAA TGTAGTGGGA GGGGAAGTGG ACACAAATCA
CAATGAAGAA GATGATGAAG AGCCCATCCC TGAGTCCAGC
GAGCTGACAC TTCAGGAACT TTTGGGAGAA GAAAGAAGAA
ACAAGAAAGG TCCTCGAGTG GACCCCCTGG AAACTGAACT
TGGTGTTAAA ACCCTGGATT GTCGAAAACC ACTTATCCCT
TTTGAAGAGT TTATTAATGA ACCACTGAAT GAGGTTCTAG
AAATGGATAA AGATTATACT TTTTTCAAAG TAGAAACAGA
GAACAAATTC TCTTTTATGA CATGTCCCTT TATATTGAAT
GCTGTCACAA AGAATTTGGG ATTATATTAT GACAATAGAA
TTCGCATGTA CAGTGAACGA AGAATCACTG TTCTCTACAG
CTTAGTTCAA GGACAGCAGT TGAATCCATA TTTGAGACTC
AAAGTTAGAC GTGACCATAT CATAGATGAT GCACTTGTCC
GGCTAGAGAT GATCGCTATG GAAAATCCTG CAGACTTGAA
GAAGCAGTTG TATGTGGAAT TTGAAGGAGA ACAAGGAGTT
GATGAGGGAG GTGTTTCCAA AGAATTTTTT CAGCTGGTTG
TGGAGGAAAT CTTCAATCCA GATATTGGTA TGTTCACATA
CGATGAATCT ACAAAATTGT TTTGGTTTAA TCCATCTTCT
TTTGAAACTG AGGGTCAGTT TACTCTGATT GGCATAGTAC
TGGGTCTGGC TATTTACAAT AACTGTATAC TGGATGTACA
TTTTCCCATG GTTGTCTACA GGAAGCTAAT GGGGAAAAAA
GGAACTTTTC GTGACTTGGG AGACTCTCAC CCAGTTCTAT
ATCAGAGTTT AAAAGATTTA TTGGAGTATG AAGGGAATGT
GGAAGATGAC ATGATGATCA CTTTCCAGAT ATCACAGACA
GATCTTTTTG GTAACCCAAT GATGTATGAT CTAAAGGAAA
ATGGTGATAA AATTCCAATT ACAAATGAAA ACAGGAAGGA
ATTTGTCAAT CTTTATTCTG ACTACATTCT CAATAAATCA
GTAGAAAAAC AGTTCAAGGC TTTTCGGAGA GGTTTTCATA
TGGTGACCAA TGAATCTCCC TTAAAGTACT TATTCAGACC
AGAAGAAATT GAATTGCTTA TATGTGGAAG CCGGAATCTA
GATTTCCAAG CACTAGAAGA AACTACAGAA TATGACGGTG
GCTATACCAG GGACTCTGTT CTGATTAGGG AGTTCTGGGA
AATCGTTCAT TCATTTACAG ATGAACAGAA AAGACTCTTC
TTGCAGTTTA CAACGGGCAC AGACAGAGCA CCTGTGGGAG
GACTAGGAAA ATTAAAGATG ATTATAGCCA AAAATGGCCC
AGACACAGAA AGGTTACCTA CATCTCATAC TTGCTTTAAT
GTGCTTTTAC TTCCGGAATA CTCAAGCAAA GAAAAACTTA
AAGAGAGATT GTTGAAGGCC ATCACGTATG CCAAAGGATT
TGGCATGCTG TAAAACAAAA CAAAACAAAA TAAAACAAAA
AAAAGGAAGG
(AK292514.1).

### Example 6—In Vitro Testing of Human UBE3A Vector Construct

Human vector properties were tested in HEK293 cells (American Type Culture Collection, Manassas, Va.), grown at 37° C. 5% CO₂ in DMEM with 10% FBS and 1% Pen/Strep and subcultured at 80% confluence.

The vector (2 μg/well in a 6-well plate) was transfected into the cells using PEI transfection method. The cells were subcultured at 0.5×10⁶ cells per well in a 6-well plate with DMEM medium two days before the transfection. Medium was replaced the night before transfection. Endotoxin-free dH₂O was heated to at around 80° C., and polyethylenimine (Sigma-Aldrich Co. LLC, St. Louis, Mo.) dissolved. The solution was allowed to cool to around 25° C., and the solution neutralized using sodium hydroxide. AAV4-STUb vector or negative control (medium only) was added to serum-free DMEM at 2 μg to every 200 μl for each well transfected, and 9p of 1 μg/μl polyethylenimine added to the mix for each well. The transfection mix was incubated at room temperature for 15 minutes, then added to each well of cells at 210 μl per well and incubated for 48 hours. Cells and media were harvested by scraping the cells from the plates. The medium and cells were then centrifuged at 5000×g for 5 minutes.

For Western blotting of the extracts, cell pellets were resuspended in 50 μL of hypo-osmotic buffer and the cells lysed by three repeated freeze/thaws. 15 μL of lysate was heated with Lamelli sample buffer and run on a BioRad 4-20% acrylamide gel. Transferred to nitrocellulose membrane using a TransBlot. The blot was blocked with 5% milk and protein detected using an anti-E6AP antibody.

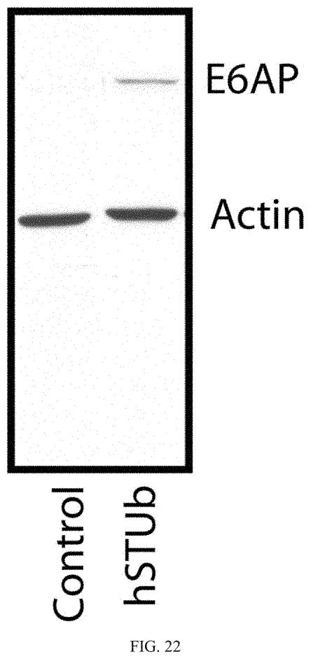

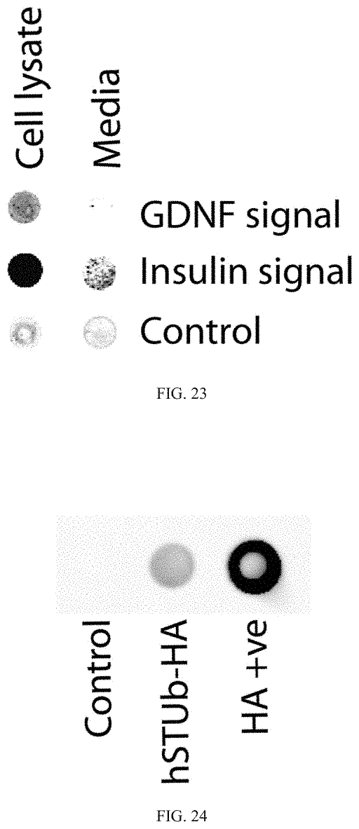

As seen in FIG. 22, cells transfected with the construct express the UBE3A gene, i.e. E6-AP. Furthermore, appending the gene to the various secretion signals exhibited mixed results, based on the secretion signal peptide. For example, transfection using constructs based on the GDNF secretion signal exhibited less expression and no detectable secretion from the transfected cells, as seen in FIG. 23. Use of the insulin secretion signal resulted in moderate secretion of E6AP from transfected cells, along with high expression of the construct within the cell. The results of insulin-signal secretion were confirmed using an HA-tagged construct, as seen in FIG. 24.

### Example 7—Efficacy of Secretion Peptides

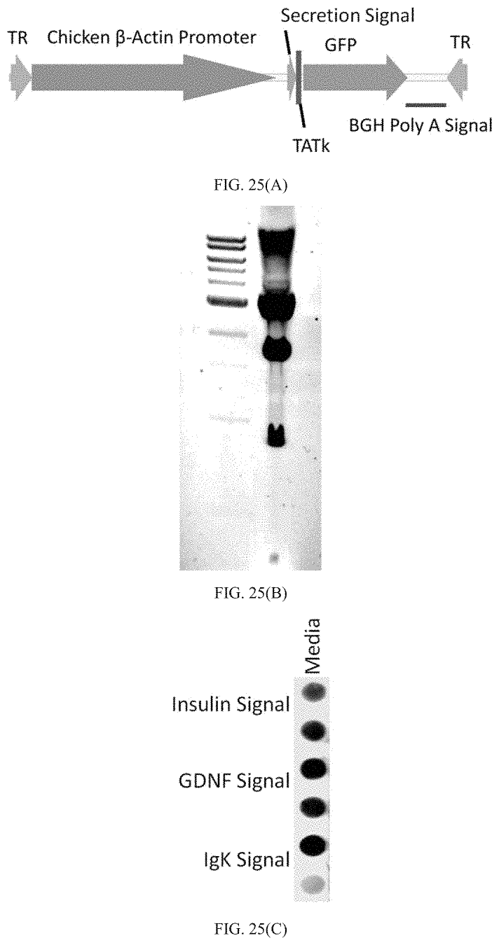

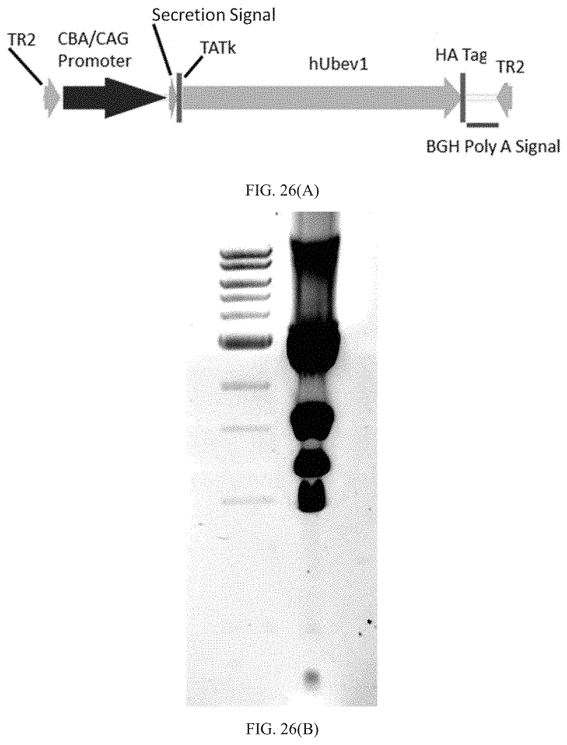

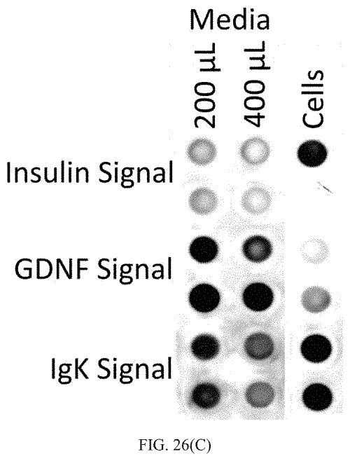

The efficacy of secretion peptides in promoting extracellular secretion of the protein by neurons was measured by creating plasmid constructs containing the various secretion signals, GFP or a human Ube3A version 1 (hUbev1) gene, and the CPP TATk, as seen in FIGS. 25(A) and 26(A). GFP was generated to use as a reporter gene for in vivo testing and to act as a control to hUbev1 in future AS studies. The secretion signals tested in this experiment were GDNF secretion signal, human insulin secretion signal, and IgK secretion signal. The amino acid sequences for the secretion signals are as follows;

for insulin:
(SEQ ID NO: 18)
MALWMRLLPLLALLALWGPDPAAA
(CAA08766.1);
for GDNF:
(SEQ ID NO: 3)
MKLWDVVAVCLVLLHTASA;
for IgK:
(SEQ ID NO: 19)
METDTLLLWVLLLWVPGSTG
(AAH80787.1).

The plasmid constructs containing the various secretion signals were generated and gel electrophoresis run to confirm successful gene insertion for each plasmid. As seen in FIGS. 25(B) and 26(B), both GFP and hUbev1 were successfully integrated into the plasmids. The efficacy of the selected secretion signals in inducing secretion of peptide by neurons was measured by transfecting the plasmid constructs into HEK293 cells and measuring the concentration of GFP in the media via dot blot. Extracts from the media were collected and X μl were placed onto nitrocellulose paper, followed by immunostaining. The results indicate that insulin signal resulted in moderate extracellular protein levels, and strong to high extracellular protein levels with IgK and GDNF signals, as seen in FIGS. 25(C) and 26(C). Thus, each signal is effective at inducing secretion of peptide in neurons, and that the hUbev1/GDNF signal-containing plasmid was particularly effective at inducing secretion of E6-AP.

### Example 8—Efficacy of Cell Penetrating Peptide

The efficacy of the select CPP signals in inducing reuptake of the protein by neurons was measured by creating plasmid constructs containing the secretion signal (GDNF), the hUbev1 gene, and the various CPP signals, outlined below, and transfecting them into HEK293 cells.

| (SEQ ID NO: 20) | |
|---|---|
| for penetratin: | RQIKIWFQNRRMKWKK; |
| (SEQ ID NO: 12) | |
| for TATk: | YARKAARQARA; |
| (SEQ ID NO: 21) | |
| for R6W3: | RRWWRRWRR; |
| (SEQ ID NO: 22) | |
| for pVEC | LLIILRRRIRKQAHAHSK. |

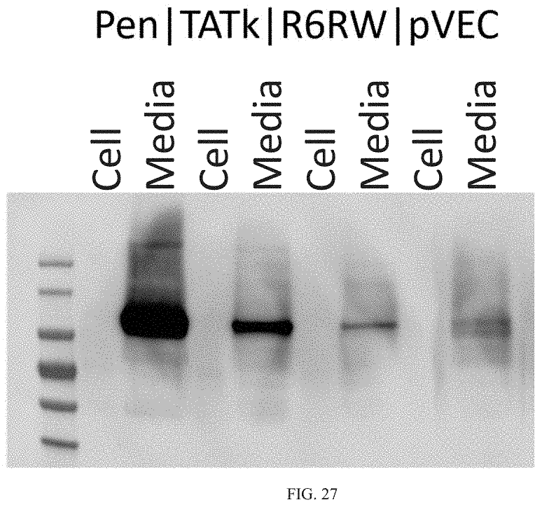

The cell lyses from these cells was then taken and added to new cell cultures of HEK293 cells and the concentration of E6-AP in these cells after incubation measured via Western blot. Results of the uptake for the CPP signals penetratin, TATk, R6RW, and pVEC are seen in FIG. 27.

### Example 9—In Vivo Testing of Human UBE3A Vector Construct in Mouse Model

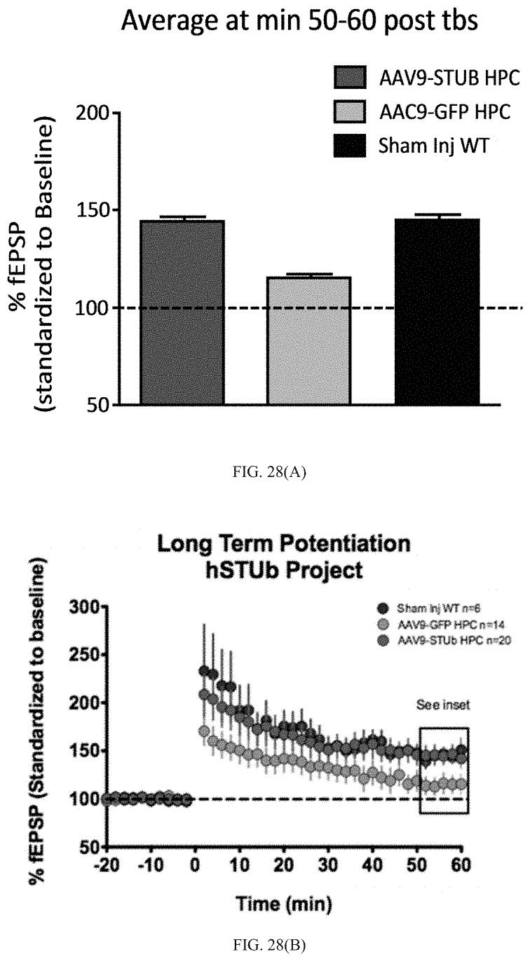

To ensure that the Ube3A gene modified to include secretion and reuptake signals maintained its ability to improve cognitive deficits associated with AS, a plasmid construct (hSTUb) containing human Ube3A version 1 (hUbev1), a secretion signal, and the CPP TATk was transduced via an rAAV vector into mouse models of AS. Long-term potentiation of the murine brain was measured via electrophysiology post-mortem and compared to GFP-transfected AS model control mice and wild-type control mice. The results indicate that the hSTUb plasmid successfully rescued LTP deficits, as seen in FIGS. 28(A) and (B).

### Example 10—Human UBE3A Vector Construct as Gene Therapy in Mouse Model

The potential of secretion and CPP signal peptides were analyzed for their ability to promote greater global distribution of E6-AP in neurons for use in a gene therapy for AS. Rescue of LTP by the hSTUb plasmid in the mouse model suggests that the UBE3A gene retains its efficacy in treating cognitive deficits in AS following the addition of secretion and CPP signals, supporting the potential of the construct in a gene therapy. The GDNF signal presents as the optimal signal for utilization in this proposed therapy as indicated by its plasmid construct showing the most secretion of E6-AP into media following transduction. Failure of the CPP signals to induce measurable reuptake of E6-AP after the application of cell lyses to the cells may be due to several factors, including insufficient concentration of E6-AP in the lyses.

### Example 11—Prophetic Human Gene Therapy

A human child presents with severe developmental delay that becomes apparent around the age of 12 months. The child later presents with absent speech, seizures, hypotonia, ataxia and mricrocephaly. The child moves with a jerky, puppet like gait and displays an unusually happy demeanor that is accompanied by laughing spells. The child has dysmorphic facial features characterized by a prominent chin, an unusually wide smile and deep-set eyes. The child diagnoses with Angelman's Syndrome. The child is treated with a therapeutically effective amount of UBE3A vector which is injected bilaterally into the left and right hippocampal hemispheres of the brain. Improvement is seen in the symptoms after treatment with a decrease in seizures, increased muscle tone, increased coordination of muscle movement and improvement in speech.

The UBE3A vector is formed from cDNA cloned from a Homo sapiens UBE3A gene. The UBE3A, version 1 gene (SEQ ID No: 9) is fused to a gene encoding a secretion signaling peptide, in this case GDNF, although insulin or IgK may also be used. The construct is inserted into the hSTUb vector, under a CMV chicken-beta actin hybrid promoter or human ubiquitin c promoter. Woodchuck hepatitis post-transcriptional regulatory element (WPRE) is present to increase expression levels.

The UBE3A-seretion signal construct is attached to a cellular uptake peptide (cell penetrating peptide or CPP) such as HIV TAT or HIV TATk. The human UBE3A vector is then transformed into E. coli using the heat shock method described in Example 2. The transformed E. coli were expanded in broth containing ampicillin to select for the vector and collect large amounts of vector.

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a method of treating UBE3A deficiencies, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A UBE3A vector, comprising:
a transcription initiation sequence;
a UBE3A sequence disposed downstream of the transcription initiation sequence, or a homologous sequence;
a secretion sequence disposed downstream of the transcription initiation sequence, or a homologous sequence; and
a cell uptake sequence disposed downstream of the transcription initiation sequence, wherein the cell uptake sequence is penetrin, R6W3, pVEC, or a homologous sequence.

2. The vector of claim 1, wherein the transcription initiation sequence is a cytomegalovirus chicken-beta actin hybrid promoter or human ubiquitin c promoter.

3. The vector of claim 2, further comprising a cytomegalovirus immediate-early enhancer sequence disposed upstream of the transcription initiation sequence.

4. The vector of claim 1, further comprising a woodchuck hepatitis post-transcriptional regulatory element.

5. The vector of claim 1, further comprising a plasmid, wherein the plasmid is a recombinant adeno-associated virus serotype 2-based plasmid, and wherein the recombinant adeno-associated virus serotype 2-based plasmid lacks DNA integration elements.

6. The vector of claim 1, wherein the secretion sequence is disposed upstream of the UBE3A sequence.

7. The vector of claim 1, wherein the cell uptake sequence is disposed upstream of the UBE3A sequence and downstream of the secretion sequence.

8. The vector of claim 1, wherein the secretion sequence is insulin, GDNF, or IgK.

9. The vector of claim 1, wherein the UBE3A sequence is SEQ ID No:9, SEQ ID No:14, SEQ ID No: 15, SEQ ID No:17, a cDNA of SEQ ID No: 10, a cDNA of SEQ ID No: 16, or a homologous sequence.

10. A method of treating a neurodegenerative disorder, comprising the steps:
administering the UBE3A vector of claim 1 to a patient suffering from a neurodegenerative disorder.

11. The method of claim 10, wherein the UBE3A vector is administered to the patient via injection in a brain of the patient.

12. A composition for use in treating a neurodegenerative disorder characterized by deficient UBE3A comprising:
the UBE3A vector of claim 1; and
a pharmaceutically acceptable carrier.

13. The composition of claim 12, wherein the neurodegenerative disorder is Angelman syndrome.
